(19) United States
(12) Patent Application Publication
(10) Pub. No.: US 2009/0012087 A1
(43) Pub. Date: Jan. 8, 2009

(54) New Aza-Bicyclohexane Compounds Useful As Inhibitors Of Thrombin

(75) Inventors: Jonas Branalt, Molndal (SE); Ingemar Nilsson, Molndal (SE); Magnus Polla, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/166,407

(22) Filed: Jul. 2, 2008

Related U.S. Application Data
(60) Provisional application No. 60/947,712, filed on Jul. 3, 2007.

(51) Int. Cl. A61K 31/5377; C07D 403/08; A61K 31/41; A61K 31/4709; C07D 401/14; C07D 413/14

(52) U.S. Cl. 514/236.2; 548/254; 514/381; 544/132; 546/168; 514/314

(57) ABSTRACT

This invention relates to novel pharmaceutically useful compounds of formula (I), in particular compounds that are competitive inhibitors of trypsin-like serine proteases, especially thrombin, their use as medicaments, pharmaceutical compositions containing them and synthetic routes to their production.

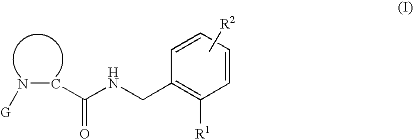

### FIELD OF THE INVENTION

This invention relates to novel pharmaceutically useful compounds, in particular compounds that are competitive inhibitors of trypsin-like serine proteases, especially thrombin, their use as medicaments, pharmaceutical compositions containing them and synthetic routes to their production.

### BACKGROUND

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V, factor VIII and factor XI leading to a “positive feedback” generation of thrombin from prothrombin.

By inhibiting the aggregation of platelets and the formation and crosslinking of fibrin, effective inhibitors of thrombin would be expected to exhibit antithrombotic activity. In addition, antithrombotic activity would be expected to be enhanced by effective inhibition of the positive feedback mechanism. Indeed, the convincing antithrombotic effects of a thrombin inhibitor in man have been described by S. Schulman et al. in N. Engl. J. Med. 349, 1713-1721 (2003), L. Wallentin et al. in Lancet 362, 789-97 (2003) and H.-C. Diener et al. in Cerebrovasc Dis 21, 279-293 (2006).

The early development of low molecular weight inhibitors of thrombin has been described by Claesson in Blood Coagul. Fibrinol. 5, 411 (1994).

Blombäck et al. (in J. Clin. Lab. Invest. 24, suppl. 107, 59 (1969)) reported thrombin inhibitors based on the amino acid sequence situated around the cleavage site for the fibrinogen Aα chain. Of the amino acid sequences discussed, these authors suggested the tripeptide sequence Phe-Val-Arg (P9-P2-P1, hereinafter referred to as the P3-P2-P1 sequence) would be the most effective inhibitor.

Thrombin inhibitors based (at the P1-position of the molecule) upon the 2-heteroaromatic substituted 1-yl-benzylamide structural unit are disclosed in U.S. Pat. No. 7,144,899 and WO2004032834.

Thrombin inhibitors based (at the P2-position of the molecule) upon the 1-acetyl-pyrrolidine-2-carboxylic acid amide, 1-acetyl-piperidine-2-carboxylic acid amide or 1-acetyl-azepane-2-carboxylic acid amide structural units are disclosed in U.S. Pat. No. 7,144,899.

Thrombin inhibitors based (at the P2-position of the molecule) upon the 1-acetyl-pyrrolidine-2-carboxylic acid amide or 1-acetyl-dihydropyrrole-2-carboxylic acid amide structural units are disclosed in U.S. Pat. No. 6,515,011 and WO2004032834.

Thrombin inhibitors based (at the P2-position of the molecule) upon the 1-acetyl-azepane-2-carboxylic acid amide structural unit are disclosed in U.S. Pat. No. 6,528,503.

Thrombin inhibitors based (at the P2-position of the molecule) upon the aza-bicyclo[3.1.0]hexane-1-carboxylic acid amide structural unit are disclosed in U.S. Pat. No. 6,288,077.

There remains a need for effective inhibitors of trypsin-like serine proteases, such as thrombin. There is also a need for compounds that have a favourable pharmacokinetic profile. Such compounds would be expected to be useful as anticoagulants and therefore in the therapeutic treatment of thrombosis and related disorders.

### DISCLOSURE OF THE INVENTION

In one aspect of the present invention there is provided a compound of formula (I)

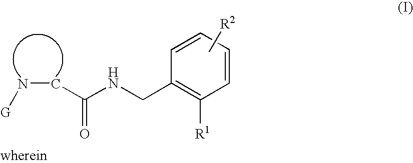

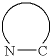

forms an aza-bicyclo[3.1.0]hexane, or

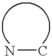

forms an aza-bicyclo[2.1.1]hexane;

R¹ is a 5-membered heteroaryl ring containing 2, 3 or 4 heteroatoms, selected from N, O and S, wherein at least 2 heteroatoms are N, and 0 or 1 heteroatoms are O or S, wherein said 5-membered heteroaryl ring is substituted, at any carbon ring atom, by 0, 1 or 2 substituents independently selected from C₁₋₆ alkyl or a 6-membered heteroaryl ring containing 1 or 2 nitrogen atoms, wherein said 6-membered heteroaryl ring is substituted, at any carbon ring atom, by 0, 1, 2 or 3 substituents independently selected from C₁₋₆ alkyl;

R² is H, halogen, cyano, C₁₋₆ alkyl or C₁₋₆ alkoxy, wherein said C₁₋₆ alkyl or C₁₋₆ alkoxy is substituted by 0, 1, 2, 3, 4 or 5 halogen;

G represents

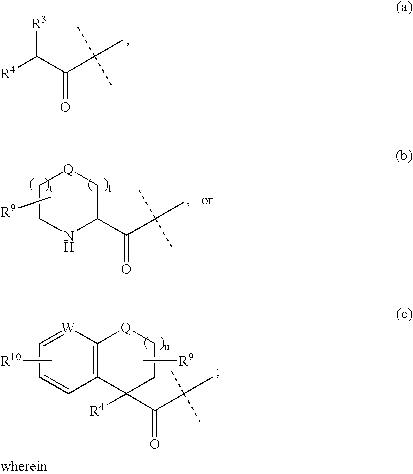

R³ is H, R⁵, C₁₋₆ alkyl, C₂₋₆ alkenyl, C₂₋₆ alkynyl or C₃₋₆ cycloalkyl, wherein each of said C₁₋₆ alkyl, C₂₋₆ alkenyl, C₂₋₆ alkynyl and C₃₋₆ cycloalkyl are independently substituted by 0, 1, 2, 3, 4 or 5 substituents selected from halogen or 0, 1 or 2 substituents selected from OH, oxo, cyano, NH₂, NH(C₁₋₄ alkyl), N(C₁₋₄ alkyl)₂, C₁₋₄ alkyl, C₃₋₆ cycloalkyl, C₄₋₇ cycloalkenyl, cycloheteroalkyl, R⁵ or R⁶;

R⁵ is phenyl,

a 5 or 6-membered heteroaromatic ring containing 1, 2 or 3 heteroatoms independently selected from O, S or N,

a 4-, 5- or 6-membered cycloheteroalkyl ring containing 1 or 2 heteroatoms independently selected from O, S or N or

a phenyl-fused 5- or 6-membered cycloheteroalkyl ring containing 1 or 2 heteroatoms independently selected from O, S or N, wherein said phenyl, said heteroaromatic ring, said cycloheteroalkyl ring and said phenyl-fused cycloheteroalkyl ring are substituted, at any carbon ring atom, by 0, 1, 2, 3, 4 or 5 substituents independently selected from COOH, OH, halogen, CF₃, CHF₂, CH₂F, cyano, C₁₋₆ alkyl, R⁶ or SO₂R⁷;

R⁶ is C₁₋₆ alkoxy, wherein said C₁₋₆ alkoxy is substituted by 0, 1, 2, 3, 4 or 5 halogen;

R⁷ is C₁₋₆ alkyl;

R⁴ is OH or NHR⁸, wherein R⁸ is H or SO₂R⁷ wherein said R⁷ is substituted by 0, 1, 2 or 3 substituents independently selected from OH, halogen, cyano, R⁶, or C₃₋₇ cycloalkyl;

Q is O, CH₂ or S(O)ₙ;

W is C or N;

n is independently 0, 1 or 2;

t is independently 0, 1 or 2;

u is independently 0 or 1;

R⁹ is 0, 1, 2, 3, 4 or 5 substituents selected from halogen, OH, oxo, cyano, C₁₋₄ alkyl, C₃₋₆ cycloalkyl, R⁵ or R⁶, wherein said C₁₋₄ alkyl is substituted by 0 or 1 substituent selected from R⁵, NH₂, NH(C₁₋₄ alkyl) or N(C₁₋₄ alkyl)₂; and

R¹⁰ is 0, 1, 2, 3, 4 or 5 substituents selected from halogen, OH, cyano, C₁₋₄ alkyl, C₃₋₆ cycloalkyl, R⁵ or R⁶, wherein said C₁₋₄ alkyl is substituted by 0 or 1 substituent selected from R⁵, NH₂, NH(C₁₋₄ alkyl) or N(C₁₋₄ alkyl)₂;

or a pharmaceutically acceptable salt or an enantiomer or a pharmaceutically acceptable salt of said enantiomer.

In a further aspect of the invention there is provided pharmaceutical formulations comprising a therapeutically effective amount of the compound of formula (I) and a pharmaceutically acceptable diluent, excipients and/or inert carrier.

In yet a further aspect of the invention there is provided a pharmaceutical formulation comprising the compound of formula (I) for use in the treatment of those conditions where inhibition of thrombin is beneficial, such as thrombo-embolism and/or conditions where anticoagulant therapy is indicated.

In still a further aspect of the invention there is provided the compound of formula (I) for use in therapy, especially for the treatment of conditions where inhibition of thrombin is beneficial, such as thrombo-embolism and/or conditions where anticoagulant therapy is indicated.

In another aspect of the invention there is provided a process for the preparation of compounds of formula (I), and the intermediates used in the preparation thereof.

These and other aspects of the present invention are described in greater detail herein below.

### DETAILED DESCRIPTION OF THE INVENTION

The object of the present invention is to provide compounds that are competitive inhibitors of trypsin-like serine proteases, especially thrombin, their use as medicaments, pharmaceutical compositions containing them and synthetic routes to their production.

Listed below are definitions of various terms used in the specification and claims to describe the present invention.

For the avoidance of doubt it is to be understood that where in this specification a group is qualified by “hereinbefore defined”, “defined hereinbefore” or “defined above” the said group encompasses the first occurring and broadest definition as well as each and all of the other definitions for that group.

For the avoidance of doubt it is to be understood that in this specification “C₁₋₆” means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl.

In this specification, unless stated otherwise, the term “cycloalkyl” refers to a saturated cyclic hydrocarbon ring system. The term “C₃₋₆ cycloalkyl” may be cyclopropyl, cyclobutyl, cyclopentyl or cyclohexyl.

In this specification, unless stated otherwise, the term “alkenyl” includes both straight and branched chain alkenyl groups. The term C₂₋₆ alkenyl having 2 to 6 carbon atoms and one or two double bonds, and may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term C₂₋₆ alkynyl having 2 to 6 carbon atoms and one or two triple bonds, and may be, but is not limited to etynyl, propargyl, pentynyl or hexynyl and a butynyl group may for example be butyn-3-yl or butyn-4-yl.

In this specification, unless stated otherwise, the term “cycloalkenyl” refers to a non-aromatic cyclic hydrocarbon ring system containing one or two double bonds. The term “C₄₋₇ cycloalkenyl” may be, but is not limited to cyclobutenyl, cyclopentenyl, cyclohexenyl or cycloheptenyl and a cyclopentenyl group may for example be cyclopenten-3-yl or cyclopenten-4-yl.

In this specification, unless stated otherwise, the term “alkoxy” includes both straight or branched alkoxy groups. C₁₋₆ alkoxy may be, but is not limited to methoxy, ethoxy, n-propoxy, i-propoxy, n-butoxy, i-butoxy, s-butoxy, t-butoxy, n-pentoxy, i-pentoxy, t-pentoxy, neo-pentoxy, n-hexyloxy, i-hexyloxy or t-hexyloxy.

In this specification, unless stated otherwise, the term “5-membered heteroaryl ring containing 2, 3 or 4 heteroatoms, selected from N, O and S, wherein at least 2 heteroatoms are N, and 0 or 1 heteroatoms are O or S” includes aromatic heterocyclic rings. Examples of such rings are imidazole, tetrazole, triazole, thiadiazole or oxadiazole.

In this specification, unless stated otherwise, the term “6-membered heteroaryl ring containing 1 or 2 nitrogen atoms” includes pyridine, pyridazine, pyrimidine or pyrazine.

In this specification, unless stated otherwise, the term “4-, 5- or 6-membered cycloheteroalkyl ring having 1 or 2 heteroatoms selected from O, S and N” includes oxetane, azetidine, oxazetidine, pyrrolidine, imidazoline, tetrahydrofuran, oxazolidine, piperidine, piperazine, hexahydropyridazine, hexahydropyrimidine, morpholine, oxazinane, thietane, thietane 1-oxide, thietane 1,1-dioxide, tetrahydra-thiophene, tetrahydra-thiophene 1-oxide, tetrahydra-thiophene 1,1-dioxide, tetrahydra-thiopyran, tetrahydra-thiopyran 1-oxide or tetrahydra-thiopyran 1,1-dioxide.

In this specification, unless stated otherwise, the term “5 or 6-membered heteroaromatic ring containing 1, 2 or 3 heteroatoms independently selected from O, S or N” includes furan, thiophene, pyrrole, oxazole, isoxazole, thiazole, isothiazole, imidazole, triazole, thiadiazole, oxadiazole, pyridine, pyridazine, pyrimidine, pyrazine or triazine.

In this specification, unless stated otherwise, the term “phenyl-fused 5- or 6-membered cycloheteroalkyl ring containing 1 or 2 heteroatoms independently selected from O, S or N” includes indoline, dihydroisoindole, dihydrobenzofuran, dihydroisobenzofuran, dihydrobenzothiophene, dihydrobenzoimidazole, dihydroindazole, dihydrobenzooxazole, dihydrobenzothiazole, tetrahydroquinoline, tetrahydroisoquinoline, tetrahydroquinoxaline, tetrahydraquinazoline, tetrahydrophtalazine, chroman, isochroman, thiochroman, isothiochroman, dihydrobenzooxazine or dihydrobenzothiazine.

In this specification, unless stated otherwise, the term “halogen” may be fluoro, chloro, bromo or iodo.

In this specification,

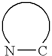

represents motifs of the following structures

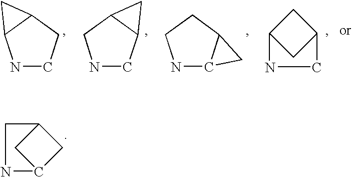

In one embodiment of the invention R¹ is a 5-membered heteroaryl ring containing 2, 3 or 4 heteroatoms, selected from N, O and S, wherein at least 2 heteroatoms are N, and 0 or 1 heteroatoms are O or S, wherein said 5-membered heteroaryl ring is substituted, at any carbon ring atom, by 0, 1 or 2 substituents independently selected from C₁₋₆ alkyl or a 6-membered heteroaryl ring containing 1 or 2 nitrogen atoms, wherein said 6-membered heteroaryl ring is substituted, at any carbon ring atom, by 0, 1, 2 or 3 substituents independently selected from C₁₋₆ alkyl.

In a further embodiment of the invention R¹ is a 5-membered heteroaryl ring containing 2, 3 or 4 heteroatoms, selected from N, O and S, wherein at least 2 heteroatoms are N, and 0 or 1 heteroatoms are O or S.

In a further embodiment of the invention R¹ is triazole.

In a further embodiment of the invention R¹ is tetrazole.

In one embodiment of the invention R² is H, halogen, cyano, C₁₋₆ alkyl or C₁₋₆ alkoxy, wherein said C₁₋₆ alkyl or C₁₋₆ alkoxy is substituted by 0, 1, 2, 3, 4 or 5 halogen. In a further embodiment of the invention R² is H or halogen. In still another embodiment of the invention R² is H, Cl or F.

In one embodiment of the invention

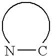

forms an aza-bicyclo[3.1.0]hexane,

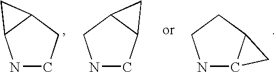

In another embodiment of the invention

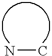

forms an aza-bicyclo[2.1.1]hexane,

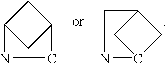

In still a further embodiment of the invention the stereochemical configuration around the carbon in the aza-bicyclo[3.1.0]hexane or aza-bicyclo[2.1.1]hexane which is covalently bound to the carbonyl is (S).

In one embodiment of the invention G is

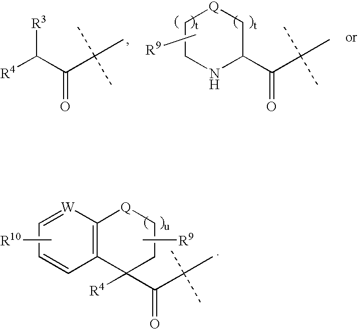

In a further embodiment of the invention G is

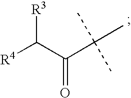

R³ is H, R⁵, C₁₋₆ alkyl, C₂₋₆ alkenyl, C₂₋₆ alkynyl or C₃₋₆ cycloalkyl, wherein each of said C₁₋₆ alkyl, C₂₋₆ alkenyl, C₂₋₆ alkynyl, and C₃₋₆ cycloalkyl are independently substituted by 0, 1, 2, 3, 4 or 5 substituents selected from halogen or 0, 1 or 2 substituents selected from OH, oxo, cyano, NH₂, NH(C₁₋₄ alkyl), N(C₁₋₄ alkyl)₂, C₁₋₄ alkyl, C₃₋₆ cycloalkyl, C₄₋₇ cycloalkenyl, cycloheteroalkyl, R⁵ or R⁶,

wherein R⁵ is phenyl, a 5 or 6-membered heteroaromatic ring containing 1, 2 or 3 heteroatoms independently selected from O, S or N, a 4-, 5- or 6-membered cycloheteroalkyl ring containing 1 or 2 heteroatoms independently selected from O, S or N or a phenyl-fused 5- or 6-membered cycloheteroalkyl ring containing 1 or 2 heteroatoms independently selected from O, S or N, wherein said phenyl, said heteroaromatic ring, said cycloheteroalkyl ring and said phenyl-fused cycloheteroalkyl ring are substituted, at any carbon ring atom, by 0, 1, 2, 3, 4 or 5 substituents independently selected from COOH, OH, halogen, CF₃, CHF₂, CH₂F, cyano, C₁₋₆ alkyl, R⁶ or SO₂R⁷;
R⁶ is C₁₋₆ alkoxy, wherein said C₁₋₆ alkoxy is substituted by 0, 1, 2, 3, 4 or 5 halogen;
R⁷ is C₁₋₆ alkyl; and

R⁴ is OH or NHR⁸, wherein R⁸ is H or SO₂R⁷ wherein said R⁷ is substituted by 0, 1, 2 or 3 substituents independently selected from OH, halogen, cyano, R⁶, COOH, C₃₋₇ cycloalkyl, SO₂R⁷ or COOR⁷;
wherein R⁶ is C₁₋₆ alkoxy, wherein said C₁₋₆ alkoxy is substituted by 0, 1, 2, 3, 4 or 5 halogen; and
R⁷ is C₁₋₆ alkyl.

In a further embodiment of the invention G is

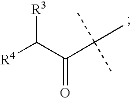

R³ is C₁₋₆ alkyl, C₃₋₆ cycloalkyl, a 5 or 6-membered heteroaromatic ring containing 1, 2 or 3 heteroatoms independently selected from O, S or N,

a 4-, 5- or 6-membered cycloheteroalkyl ring containing 1 or 2 heteroatoms independently selected from O, S or N, or R¹¹, wherein said C₁₋₆ alkyl, said C₃₋₆ cycloalkyl, said heteroaromatic ring and said cycloheteroalkyl ring are substituted by 0 or 1 substituents selected from NH₂, NH(C₁₋₄ alkyl), N(C₁₋₄ alkyl)₂, C₃ cycloalkyl, R⁶ or R¹¹,
wherein R⁶ is C₁₋₆ alkoxy, wherein said C₁₋₆ alkoxy is substituted by 0, 1, 2, 3, 4 or 5 halogen;
R¹¹ is phenyl, wherein said phenyl is substituted by 0, 1 or 2 substituents selected from halogen; and

R⁴ is OH or NH₂.

In a still further embodiment of the invention G is

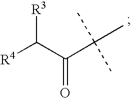

R³ is C₃₋₆ cycloalkyl, R¹¹ or C₁₋₆ alkyl, wherein said C₁₋₆ alkyl is substituted by 0 or 1 substituents selected from C₃ cycloalkyl, N(C₁₋₄ alkyl)₂, R⁶ or R¹¹,
wherein R⁶ is C₁₋₆ alkoxy, wherein said C₁₋₆ alkoxy is substituted by 0, 1, 2, 3, 4 or 5 halogen; and
R¹¹ is phenyl, wherein said phenyl is substituted by 0, 1 or 2 substituents selected from halogen; and

R⁴ is OH or NH₂.

In one embodiment of the invention the stereochemical configuration around the carbon substituted by R³ and R⁴ in G is (R).

In a further embodiment G is

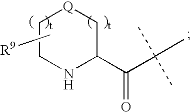

R⁹ is 0, 1, 2, 3, 4 or 5 substituents selected from halogen, OH, oxo, cyano, C₁₋₄ alkyl, C₃₋₆ cycloalkyl, R⁵ or R⁶, wherein said C₁₋₄ alkyl is substituted by 0 or 1 substituent selected from R⁵, NH₂, NH(C₁₋₄ alkyl) or N(C₁₋₄ alkyl)₂;

R⁵ is phenyl, which is substituted, by 0, 1, 2, 3, 4 or 5 substituents independently selected from COOH, OH, halogen, CF₃, cyano, C₁₋₆ alkyl, R⁶ or SO₂R⁷,
wherein R⁶ is C₁₋₆ alkoxy, wherein said C₁₋₆ alkoxy is substituted by 0, 1, 2, 3, 4 or 5 halogen; and
R⁷ is C₁₋₆ alkyl;

Q is O, CH₂ or S(O)ₙ;

n is independently 0, 1 or 2; and

t is independently 0, 1 or 2.

In a still further embodiment of the invention G is

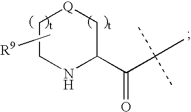

R⁹ is 0, 1 or 2 substituents selected from oxo, C₁₋₄ alkyl, R⁵ or R⁶;

R⁵ is phenyl, which is substituted, by 0, 1, 2, 3, 4 or 5 substituents independently selected from COOH, OH, halogen, CF₃, cyano, C₁₋₆ alkyl, R⁶ or SO₂R⁷;
wherein R⁶ is C₁₋₆ alkoxy, wherein said C₁₋₆ alkoxy is substituted by 0, 1, 2, 3, 4 or 5 halogen; and
R⁷ is C₁₋₆ alkyl;

Q is O or CH₂; and

t is independently 0 or 1.

In a still further embodiment of the invention G is

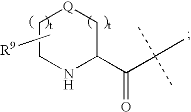

R⁹ is 0, 1 or 2 substituents selected from oxo, C₁₋₄ alkyl;

Q is O or CH₂; and

t is independently 0 or 1.

In a further embodiment of the invention G is

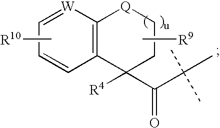

R⁴ is OH or NHR⁸, wherein R⁸ is H or SO₂R⁷ wherein said R⁷ is substituted by 0, 1, 2 or 3 substituents independently selected from OH, halogen, cyano, R⁶ or C₃₋₇ cycloalkyl;
wherein R⁶ is C₁₋₆ alkoxy, wherein said C₁₋₆ alkoxy is substituted by 0, 1, 2, 3, 4 or 5 halogen; and

R⁷ is C₁₋₆ alkyl;

R⁹ is 0, 1, 2, 3, 4 or 5 substituents selected from halogen, OH, oxo, cyano, C₁₋₄ alkyl, C₃₋₆ cycloalkyl, R⁵ or R⁶, wherein said C₁₋₄ alkyl is substituted by 0 or 1 substituent selected from R⁵, NH₂, NH(C₁₋₄ alkyl) or N(C₁₋₄ alkyl)₂;

R¹⁰ is 0, 1, 2, 3, 4 or 5 substituents selected from halogen, OH, cyano, C₁₋₄ alkyl, C₃₋₆ cycloalkyl, R⁵ or R⁶, wherein said C₁₋₄ alkyl is substituted by 0 or 1 substituents selected from R⁵, NH₂, NH(C₁₋₄ alkyl) or N(C₁₋₄ alkyl)₂;

R⁵ is phenyl, which is substituted, by 0, 1, 2, 3, 4 or 5 substituents independently selected from COOH, OH, halogen, CF₃, cyano, C₁₋₆ alkyl, R⁶ or SO₂R⁷,
wherein R⁶ is C₁₋₆ alkoxy, wherein said C₁₋₆ alkoxy is substituted by 0, 1, 2, 3, 4 or 5 halogen; and
R⁷ is C₁₋₆ alkyl;

Q is O, CH₂ or S(O)ₙ;

W is C or N;

n is independently 0, 1 or 2; and

u is independently 0 or 1.

In a still further embodiment of the invention G is

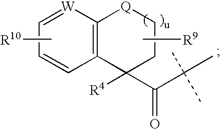

R⁴ is OH or NH₂;

R⁹ is 0, 1 or 2 substituents selected from C₁₋₄ alkyl, halogen or R⁶;

R¹⁰ is 0, 1 or 2 substituents selected from C₁₋₄ alkyl, halogen or R⁶,
wherein R⁶ is C₁₋₆ alkoxy, wherein said C₁₋₆ alkoxy is substituted by 0, 1, 2, 3, 4 or 5 halogen; and

Q is O or CH₂; and

u is independently 0 or 1.

In a still further embodiment of the invention G is

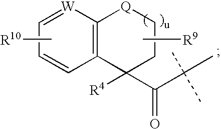

R⁴ is OH or NH₂;

R⁹ is 0, 1 or 2 substituents selected from C₁₋₄ alkyl, F, Cl, OCH₃, OCF₃, OCHF₂ or OCH₂F;

R¹⁰ is 0, 1 or 2 substituents selected from C₁₋₄ alkyl, F, Cl, OCH₃, OCF₃, OCHF₂ or OCH₂F;

Q is O or CH₂; and

u is independently 0 or 1.

In one embodiment of the invention the compound of formula (I) is selected from:
(1S,2S,5R)-3-((R)-2-Hydroxy-3,3-dimethyl-butyryl)-3-aza-bicyclo[3.1.0]hexane-2-carboxylic acid 5-chloro-2-tetrazol-1-yl-benzylamide,
(1S,2S,5R)-3-((R)-2-Hydroxy-2-phenyl-acetyl)-3-aza-bicyclo[3.1.0]hexane-2-carboxylic acid 5-chloro-2-tetrazol-1-yl-benzylamide,
(1R,2S,5S)-3-((R)-2-Hydroxy-2-phenyl-acetyl)-3-aza-bicyclo[3.1.0]hexane-2-carboxylic acid 5-chloro-2-tetrazol-1-yl-benzylamide,
(1R,3S,5R)-2-((R)-2-Hydroxy-2-phenyl-acetyl)-2-aza-bicyclo[3.1.0]hexane-3-carboxylic acid 5-chloro-2-tetrazol-1-yl-benzylamide,
(1S,3S,5S)-2-((R)-2-Hydroxy-2-phenyl-acetyl)-2-aza-bicyclo[3.1.0]hexane-3-carboxylic acid 5-chloro-2-tetrazol-1-yl-benzylamide,
(1R,2S,5S)-3-((R)-2-Hydroxy-3,3-dimethyl-butyryl)-3-aza-bicyclo[3.1.0]hexane-2-carboxylic acid 5-chloro-2-tetrazol-1-yl-benzylamide,
(1R,3S,5R)-2-((R)-2-Hydroxy-3,3-dimethyl-butyryl)-2-aza-bicyclo[3.1.0]hexane-3-carboxylic acid 5-chloro-2-tetrazol-1-yl-benzylamide,
(1S,3S,5S)-2-((R)-2-Hydroxy-3,3-dimethyl-butyryl)-2-aza-bicyclo[3.1.0]hexane-3-carboxylic acid 5-chloro-2-tetrazol-1-yl-benzylamide,
(1R,2S,5S)-3-(2-Hydroxy-hexanoyl)-3-aza-bicyclo[3.1.0]hexane-2-carboxylic acid 5-chloro-2-tetrazol-1-yl-benzylamide,
(1R,3S,5R)-2-(2-Hydroxy-hexanoyl)-2-aza-bicyclo[3.1.0]hexane-3-carboxylic acid 5-chloro-2-tetrazol-1-yl-benzylamide,
(1S,3S,5S)-2-(2-Hydroxy-hexanoyl)-2-aza-bicyclo[3.1.0]hexane-3-carboxylic acid 5-chloro-2-tetrazol-1-yl-benzylamide,
(1R,3S,5R)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide,
(1S,3S,5S)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide,
(1S,2S,5R)-3-((R)-2-Hydroxy-4,4-dimethyl-pentanoyl)-3-aza-bicyclo[3.1.0]hexane-2-carboxylic acid 5-chloro-2-tetrazol-1-yl-benzylamide,
(1S,2S,5R)-3-((R)-3-Cyclopropyl-2-hydroxy-propionyl)-3-aza-bicyclo[3.1.0]hexane-2-carboxylic acid 5-chloro-2-tetrazol-1-yl-benzylamide,
(1R,3S,5R)-2-((R)-3-Cyclopropyl-2-hydroxy-propionyl)-2-aza-bicyclo[3.1.0]hexane-3-carboxylic acid 5-chloro-2-tetrazol-1-yl-benzylamide,
(1S,3S,5S)-2-((R)-3-Cyclopropyl-2-hydroxy-propionyl)-2-aza-bicyclo[3.1.0]hexane-3-carboxylic acid 5-chloro-2-tetrazol-1-yl-benzylamide,
(1S,2S,5R)-3-((R)-2-Amino-4,4-dimethyl-pentanoyl)-3-aza-bicyclo[3.1.0]hexane-2-carboxylic acid 5-chloro-2-tetrazol-1-yl-benzylamide,
(1R,3S,5R)-2-((R)-2-Amino-4,4-dimethyl-pentanoyl)-2-aza-bicyclo[3.1.0]hexane-3-carboxylic acid 5-chloro-2-tetrazol-1-yl-benzylamide,
(1S,3S,5S)-2-((R)-2-Amino-4,4-dimethyl-pentanoyl)-2-aza-bicyclo[3.1.0]hexane-3-carboxylic acid 5-chloro-2-tetrazol-1-yl-benzylamide,
(1S,2S,5R)-3-((R)-2-Cyclohexyl-2-hydroxy-acetyl)-3-aza-bicyclo[3.1.0]hexane-2-carboxylic acid 5-chloro-2-tetrazol-1-yl-benzylamide,
(1R,3S,5R)-2-((R)-2-Cyclohexyl-2-hydroxy-acetyl)-2-aza-bicyclo[3.1.0]hexane-3-carboxylic acid 5-chloro-2-tetrazol-1-yl-benzylamide,
(1S,2S,5R)-3-((R)-3-tert-Butoxy-2-hydroxy-propionyl)-3-aza-bicyclo[3.1.0]hexane-2-carboxylic acid 5-chloro-2-tetrazol-1-yl-benzylamide,
(1R,3S,5R)-2-((R)-3-tert-Butoxy-2-hydroxy-propionyl)-2-aza-bicyclo[3.1.0]hexane-3-carboxylic acid 5-chloro-2-tetrazol-1-yl-benzylamide,
(1S,2S,5R)-3-((R)-2-Hydroxy-3-phenyl-propionyl)-3-aza-bicyclo[3.1.0]hexane-2-carboxylic acid 5-chloro-2-tetrazol-1-yl-benzylamide,
(1R,2S,5S)-3-((R)-2-Hydroxy-3-phenyl-propionyl)-3-aza-bicyclo[3.1.0]hexane-2-carboxylic acid 5-chloro-2-tetrazol-1-yl-benzylamide,
(1S,5R)-2-((R)-2-Cyclohexyl-2-hydroxy-acetyl)-2-aza-bicyclo[3.1.0]hexane-1-carboxylic acid-5-chloro-2-tetrazol-1-yl-benzylamide,
2-((R)-2-Hydroxy-4,4-dimethyl-pentanoyl)-2-aza-bicyclo[2.1.1]hexane-1-carboxylic acid 5-chloro-2-tetrazol-1-yl-benzylamide,
2-((R)-2-Hydroxy-3-phenyl-propionyl)-2-aza-bicyclo[2.1.1]hexane-1-carboxylic acid 5-chloro-2-tetrazol-1-yl-benzylamide,
2-((R)-2-Hydroxy-2-phenyl-acetyl)-2-aza-bicyclo[2.1.1]hexane-1-carboxylic acid 5-chloro-2-tetrazol-1-yl-benzylamide,
(1S,3S,5S)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1R,2S,5S)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1S,3S,5S)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide,
(1S,2S,5R)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1S,2S,5R)-3-[2-((R)-3-Chloro-5-difluoromethoxy-phenyl)-2-hydroxy-acetyl]-3-aza-bicyclo[3.1.0]hexane-2-carboxylic acid 5-chloro-2-tetrazol-1-yl-benzylamide,
(1S,3S,5S)-2-[(R)-2-(3-Chloro-5-difluoromethoxy-phenyl)-2-hydroxy-acetyl]-2-aza-bicyclo[3.1.0]hexane-3-carboxylic acid 5-chloro-2-tetrazol-1-yl-benzylamide,
(1S,2S,5R)-3-(2-Hydroxy-hexanoyl)-3-aza-bicyclo[3.1.0]hexane-2-carboxylic acid 5-chloro-2-tetrazol-1-yl-benzylamide,
(1S,3S,5S)-2-((R)-2-Hydroxy-3-pyridin-2-yl-propionyl)-2-aza-bicyclo[3.1.0]hexane-3-carboxylic acid 5-chloro-2-tetrazol-1-yl-benzylamide,
(1R,2S,5S)-3-(2-Hydroxy-3-methoxy-3-methyl-butyryl)-3-aza-bicyclo[3.1.0]hexane-2-carboxylic acid 5-chloro-2-tetrazol-1-yl-benzylamide,
(1S,3S,5S)-2-(2-Hydroxy-3-methoxy-3-methyl-butyryl)-2-aza-bicyclo[3.1.0]hexane-3-carboxylic acid 5-chloro-2-tetrazol-1-yl-benzylamide,
(1S,3S,5S)-2-[(R)-2-(3-Chloro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-((R)-2-Amino-2-phenyl-acetyl)-2-aza-bicyclo[3.1.0]hexane-3-carboxylic acid 5-chloro-2-tetrazol-1-yl-benzylamide,
(1R,2S,5S)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide,
(1R,3S,5R)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide,
(1R,3S,5R)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-[(R)-2-Amino-2-(4-hydroxy-phenyl)-acetyl]-2-aza-bicyclo[3.1.0]hexane-3-carboxylic acid 5-chloro-2-tetrazol-1-yl-benzylamide,
(1S,3S,5S)-2-((R)-2-Amino-3-hydroxy-propionyl)-2-aza-bicyclo[3.1.0]hexane-3-carboxylic acid 5-chloro-2-tetrazol-1-yl-benzylamide,
(1R,3S,5R)-2-((R)-2-Amino-3-hydroxy-propionyl)-2-aza-bicyclo[3.1.0]hexane-3-carboxylic acid 5-chloro-2-tetrazol-1-yl-benzylamide,
(1S,3S,5S)-2-(2-Hydroxy-3-1,2,4-triazol-1-yl-propionyl)-2-aza-bicyclo[3.1.0]hexane-3-carboxylic acid 5-chloro-2-tetrazol-1-yl-benzylamide,
(1S,2S,5R)-3-(2-Hydroxy-3-1,2,4-triazol-1-yl-propionyl)-3-aza-bicyclo[3.1.0]hexane-2-carboxylic acid 5-chloro-2-tetrazol-1-yl-benzylamide,
(1S,3S,5S)-2-((R)-2-Amino-3-tert-butoxy-propionyl)-2-aza-bicyclo[3.1.0]hexane-3-carboxylic acid 5-chloro-2-tetrazol-1-yl-benzylamide,
(1S,2S,5R)-3-((R)-2-Amino-3-tert-butoxy-propionyl)-3-aza-bicyclo[3.1.0]hexane-2-carboxylic acid 5-chloro-2-tetrazol-1-yl-benzylamide,
(1S,2S,5R)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide,
(1S,3S,5S)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide,
(1S,2S,5R)-3-[(R)-2-(3-Chloro-phenyl)-2-hydroxy-acetyl]-3-aza-bicyclo[3.1.0]hexane-2-carboxylic acid 5-chloro-2-tetrazol-1-yl-benzylamide,
(1R,2S,5S)-3-[(S)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1R,3S,5R)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide,
(1R,2S,5S)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide,
(1S,2S,5R)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide,
(1S,3S,5S)-2-(4-Hydroxy-1-benzopyran-4-carbonyl)-2-aza-bicyclo[3.1.0]hexane-3-carboxylic acid 5-chloro-2-tetrazol-1-yl-benzylamide,
(rac)-2-(4-Hydroxy-1-benzopyran-4-carbonyl)-2-aza-bicyclo[2.1.1]hexane-1-carboxylic acid 5-chloro-2-tetrazol-1-yl-benzylamide,
2-((R)-2-Hydroxy-4-phenyl-butyryl)-2-aza-bicyclo[2.1.1]hexane-1-carboxylic acid 5-chloro-2-tetrazol-1-yl-benzylamide,
2-((R)-3-tert-Butoxy-2-hydroxy-propionyl)-2-aza-bicyclo[2.1.1]hexane-1-carboxylic acid 5-chloro-2-tetrazol-1-yl-benzylamide,
2-((R)-2-Cyclohexyl-2-hydroxy-acetyl)-2-aza-bicyclo[2.1.1]hexane-1-carboxylic acid 5-chloro-2-tetrazol-1-yl-benzylamide,
2-((R)-2-Amino-3-cyclohexyl-propionyl)-2-aza-bicyclo[2.1.1]hexane-1-carboxylic acid 5-chloro-2-tetrazol-1-yl-benzylamide,
(1S,3S,5S)-2-(2-Hydroxy-3-1,2,4-triazol-1-yl-propionyl)-2-aza-bicyclo[3.1.0]hexane-3-carboxylic acid 5-chloro-2-1,2,4-triazol-1-yl-benzylamide,
(1S,3S,5S)-2-[(R)-2-(3-Chloro-phenyl)-2-hydroxy-acetyl]-2-aza-bicyclo[3.1.0]hexane-3-carboxylic acid 5-fluoro-2-tetrazol-1-yl-benzylamide,
(1S,3S,5S)-2-[(R)-2-Amino-2-(3-chloro-phenyl)-acetyl]-2-aza-bicyclo[3.1.0]hexane-3-carboxylic acid 5-chloro-2-tetrazol-1-yl-benzylamide,
(1S,3S,5S)-2-[2-Amino-2-(1,1-dioxo-hexahydro-1λ⁶-thiopyran-4-yl)-acetyl]-2-aza-bicyclo[3.1.0]hexane-3-carboxylic acid 5-chloro-2-tetrazol-1-yl-benzylamide,
(1S,3S,5S)-2-[2-Amino-2-(2-fluoro-phenyl)-acetyl]-2-aza-bicyclo[3.1.0]hexane-3-carboxylic acid 5-chloro-2-tetrazol-1-yl-benzylamide,
(1S,2S,5R)-3-[2-Amino-2-(2-fluoro-phenyl)-acetyl]-3-aza-bicyclo[3.1.0]hexane-2-carboxylic acid 5-chloro-2-tetrazol-1-yl-benzylamide,
(1S,2S,5R)-3-((R)-Morpholine-3-carbonyl)-3-aza-bicyclo[3.1.0]hexane-2-carboxylic acid 5-chloro-2-tetrazol-1-yl-benzylamide,
(1S,3S,5S)-2-(5-Hydroxy-5,6,7,8-tetrahydro-quinoline-5-carbonyl)-2-aza-bicyclo[3.1.0]hexane-3-carboxylic acid 5-chloro-2-tetrazol-1-yl-benzylamide,
2-[(R)-2-Hydroxy-3-(1-methyl-cyclopropyl)-propionyl]-2-aza-bicyclo[2.1.1]hexane-1-carboxylic acid 5-chloro-2-tetrazol-1-yl-benzylamide,
2-((R)-2-Hydroxy-3,3-dimethyl-butyryl)-2-aza-bicyclo[2.1.1]hexane-1-carboxylic acid 5-chloro-2-tetrazol-1-yl-benzylamide,
(1S,5R)-2-((R)-2-Hydroxy-3,3-dimethyl-butyryl)-2-aza-bicyclo[3.1.0]hexane-1-carboxylic acid 5-chloro-2-tetrazol-1-yl-benzylamide,
2-((R)-2-Amino-3,3-dimethyl-butyryl)-2-aza-bicyclo[2.1.1]hexane-1-carboxylic acid 5-chloro-2-tetrazol-1-yl-benzylamide,
(1S,2S,5R)-3-(4-Hydroxy-chroman-4-carbonyl)-3-aza-bicyclo[3.1.0]hexane-2-carboxylic acid 5-chloro-2-tetrazol-1-yl-benzylamide,
(1S,5R)-2-((R)-2-Hydroxy-4,4-dimethyl-pentanoyl)-2-aza-bicyclo[3.1.0]hexane-1-carboxylic acid 5-chloro-2-tetrazol-1-yl-benzylamide,
(1S,5R)-2-((R)-2-Hydroxy-2-phenyl-acetyl)-2-aza-bicyclo[3.1.0]hexane-1-carboxylic acid 5-chloro-2-tetrazol-1-yl-benzylamide,
(1S,3S,5S)-2-((R)-2-Hydroxy-3-pyrazol-1-yl-propionyl)-2-aza-bicyclo[3.1.0]hexane-3-carboxylic acid 5-chloro-2-tetrazol-1-yl-benzylamide,
(1S,2S,5R)-3-((R)-2-Hydroxy-3-pyrazol-1-yl-propionyl)-3-aza-bicyclo[3.1.0]hexane-2-carboxylic acid 5-chloro-2-tetrazol-1-yl-benzylamide,
(1S,2S,5R)-3-(2-Hydroxy-3-pyridin-2-yl-propionyl)-3-aza-bicyclo[3.1.0]hexane-2-carboxylic acid 5-chloro-2-tetrazol-1-yl-benzylamide,
(1S,5R)-2-((R)-3-tert-Butoxy-2-hydroxy-propionyl)-2-aza-bicyclo[3.1.0]hexane-1-carboxylic acid 5-chloro-2-tetrazol-1-yl-benzylamide,
(1S,3S,5S)-2-(4-Hydroxy-chroman-4-carbonyl)-2-aza-bicyclo[3.1.0]hexane-3-carboxylic acid 5-fluoro-2-tetrazol-1-yl-benzylamide,
(1S,2S,5R)-3-((R)-2-Hydroxy-3,3-dimethyl-butyryl)-3-aza-bicyclo[3.1.0]hexane-2-carboxylic acid 5-chloro-2-[1,2,4]triazol-1-yl-benzylamide,
(1S,3S,5S)-2-[(R)-2-Hydroxy-3-(3-methyl-3H-imidazol-4-yl)-propionyl]-2-aza-bicyclo[3.1.0]hexane-3-carboxylic acid 5-chloro-2-tetrazol-1-yl-benzylamide,
(1S,3S,5S)-2-(2-Hydroxy-3-piperidin-4-yl-propionyl)-2-aza-bicyclo[3.1.0]hexane-3-carboxylic acid 5-chloro-2-tetrazol-1-yl-benzylamide or
(1S,3S,5S)-2-((R)-Morpholine-3-carbonyl)-2-aza-bicyclo[3.1.0]hexane-3-carboxylic acid 5-chloro-2-tetrazol-1-yl-benzylamide acetate.

In another aspect of the present invention there is provided a compound of formula (X)

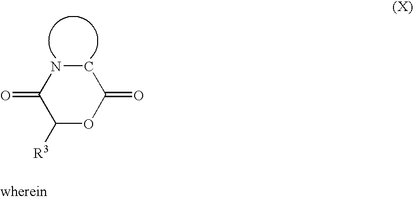

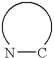

forms an aza-bicyclo[3.1.0]hexane, or

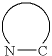

forms an aza-bicyclo[2.1.1]hexane;

R³ is C₁₋₆ alkyl, C₃₋₆ cycloalkyl, a 5 or 6-membered heteroaromatic ring containing 1, 2 or 3 heteroatoms independently selected from O, S or N,

a 4-, 5- or 6-membered cycloheteroalkyl ring containing 1 or 2 heteroatoms independently selected from O, S or N, or R¹¹, wherein said C₁₋₆ alkyl, said C₃₋₆ cycloalkyl, said heteroaromatic ring and said cycloheteroalkyl ring are substituted by 0 or 1 substituents selected from NH₂, NH(C₁₋₄ alkyl), N(C₁₋₄ alkyl)₂, C₃ cycloalkyl, R⁶ or R¹¹;

R⁶ is C₁₋₆ alkoxy, wherein said C₁₋₆ alkoxy is substituted by 0, 1, 2, 3, 4 or 5 halogen; and

R¹¹ is phenyl, wherein said phenyl is substituted by 0, 1 or 2 substituents selected from halogen or R⁶.

In yet another aspect of the present invention there is provided a compound of formula (XI)

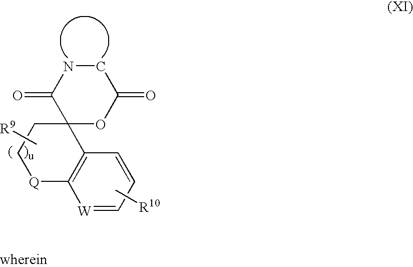

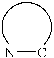

forms an aza-bicyclo[3.1.0]hexane, or

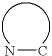

forms an aza-bicyclo[2.1.1]hexane;

R⁹ is 0, 1 or 2 substituents selected from C₁₋₄ alkyl, halogen or R⁶;

R¹⁰ is 0, 1 or 2 substituents selected from C₁₋₄ alkyl, halogen or R⁶;

R⁶ is C₁₋₆ alkoxy, wherein said C₁₋₆ alkoxy is substituted by 0, 1, 2, 3, 4 or 5 halogen;

Q is O or CH₂; and

u is independently 0 or 1.

The present invention further provides a process for the preparation of a compound of formula (I) as defined above which comprises:

(A) reacting a compound of formula (II),

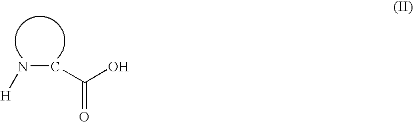

or a derivative thereof that is protected at the amino group, with an amine of formula (III)

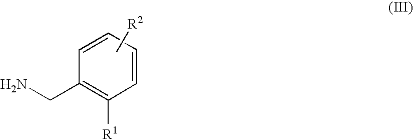

wherein R¹ and R² are as defined in formula (I) to deliver a compound of formula (IV), or a derivative thereof that is protected at the amino group,

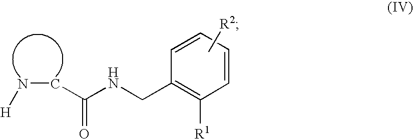

(B) reacting a compound of formula (IV),

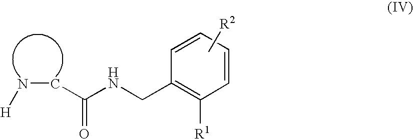

wherein R¹ and R² are as defined in formula (I), with a compound of formula (V)

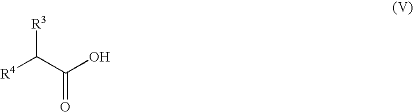

wherein R³ is as hereinbefore defined and R⁴ is OH, or a derivative thereof that is either protected at the hydroxy substituent or at both the hydroxy substituent and at the carboxylic acid, to deliver a compound of formula (I);

(C) reacting a compound of formula (IV),

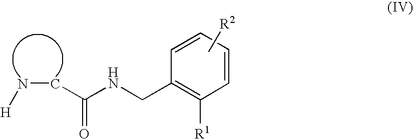

wherein R¹ and R² are as defined in formula (I), with a compound of formula (VI)

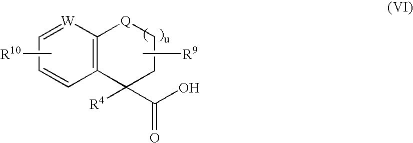

wherein R⁹, R¹⁰, W, Q and u are as hereinbefore defined and R⁴ is OH, or a derivative thereof that is either protected at the hydroxy substituent or at both the hydroxy substituent and at the carboxylic acid, to deliver a compound of formula (I);

(D) reacting a compound of formula (IV),

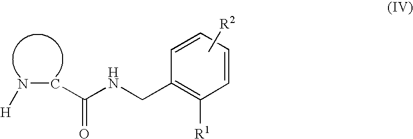

wherein R¹ and R² are as defined in formula (I), with a compound of formula (V)

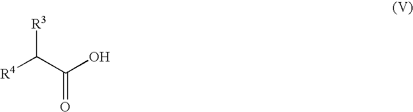

wherein R³ is as hereinbefore defined and R⁴ is NHR⁸, wherein R⁸ is as hereinbefore defined, or a derivative thereof that is protected at the amino substituent, to deliver a compound of formula (I);

(E) reacting a compound of formula (IV),

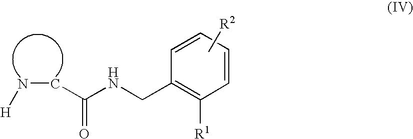

wherein R¹ and R² are as defined in formula (I), with a compound of formula (VI)

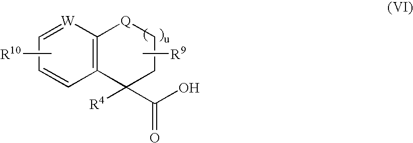

wherein R⁹, R¹⁰, W, Q and u are as hereinbefore defined and R⁴ is NHR⁸, wherein R⁸ is as hereinbefore defined, or a derivative thereof that is protected at the amino substituent, to deliver a compound of formula (I);

(F) reacting a compound of formula (IV),

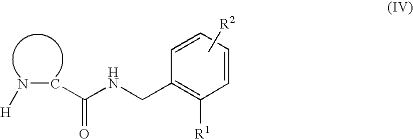

wherein R¹ and R² are as defined in formula (I), with a compound of formula (VII)

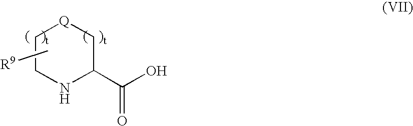

wherein R⁹, Q and t are as hereinbefore defined, or a derivative thereof that is protected at the amino group, to deliver a compound of formula (I);

(G) reacting a compound of formula (II),

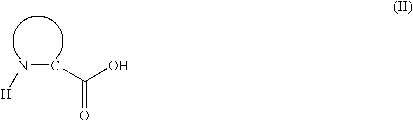

or a derivative thereof that is protected at the carboxylic acid, with a compound of formula (V)

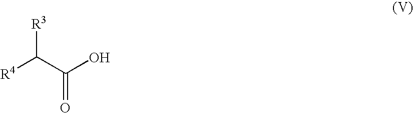

wherein R³ is as hereinbefore defined and R⁴ is OH, or a derivative thereof that is either protected at the hydroxy substituent or at both the hydroxy substituent and at the carboxylic acid, to deliver a compound of formula (VIII);

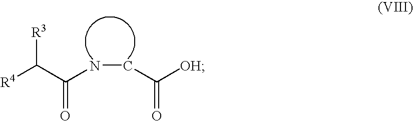

(H) reacting a compound of formula (II),

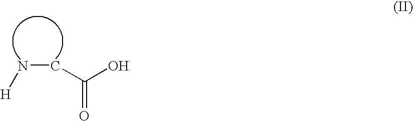

or a derivative thereof that is protected at the carboxylic acid, with a compound of formula (VI)

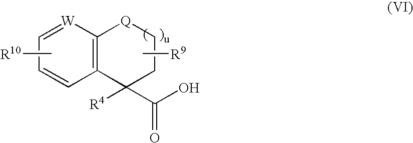

wherein R⁹, R¹⁰, W, Q and u are as hereinbefore defined and R⁴ is OH, or a derivative thereof that is either protected at the hydroxy substituent or at both the hydroxy substituent and at the carboxylic acid, to deliver a compound of formula (IX)

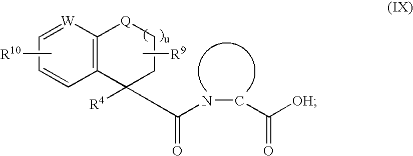

(I) reacting a compound of formula (VIII), or a derivative thereof that is protected at the carboxylic acid,

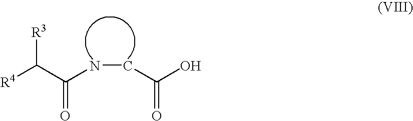

wherein R³ is as hereinbefore defined and R⁴ is OH, or a derivative thereof that is protected at the OH group, to deliver a compound of formula (X)

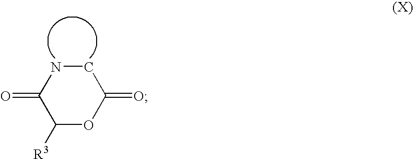

(J) reacting a compound of formula (IX), or a derivative thereof that is protected at the carboxylic acid,

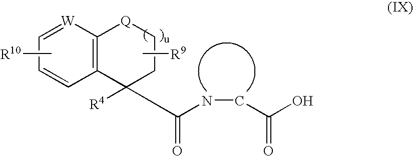

wherein R⁹, R¹⁰, W, Q and u are as hereinbefore defined and R⁴ is OH, or a derivative thereof that is protected at the OH group, to deliver a compound of formula (XI)

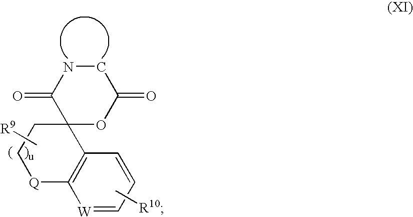

(K) reacting a compound of formula (X) or formula (XI), wherein R³, R⁹, R¹⁰, W, Q and u are as hereinbefore defined with a compound of formula (III), wherein R¹ and R² are as hereinbefore defined, to deliver a compound of formula (I);

(L) reacting a compound of formula (II),

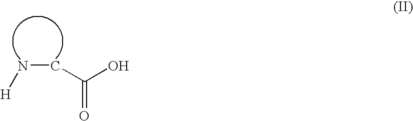

or a derivative thereof that is protected at the carboxylic acid, with a compound of formula (XII)

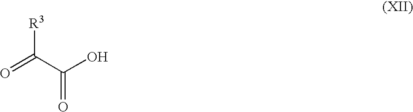

wherein R³ is as hereinbefore defined, to deliver a compound of formula (XIII)

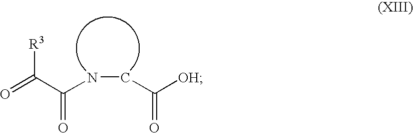

(M) reacting a compound of formula (XIII), wherein R³ is as hereinbefore defined, or a derivative thereof that is protected at the carboxylic acid, under reducing conditions to deliver a compound of formula (VIII)

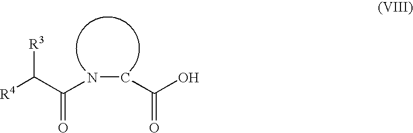

wherein R⁴ is OH, or a derivative thereof that is protected at the carboxylic acid.

Process (A) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (B) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (C) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (D) may be carried out using known procedures for preparation of amides from carboxylic acids, or analogously, e.g. as hereinafter described in the Examples. It may be carried out in a solvent, e.g. DCM, MeCN, H₂O, EtOAc or DMF, in the presence of an appropriate base, e.g. pyridine, DMAP, NMM, TEA, NaHCO₃, 2,4,6-collidine or DIPEA, and a suitable reagent, e.g. oxalyl chloride, cyanuric fluoride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (E) may be carried out using known procedures for preparation of amides from carboxylic acids, or analogously, e.g. as hereinafter described in the Examples. It may be carried out in a solvent, e.g. DCM, MeCN, H₂O, EtOAc or DMF, in the presence of an appropriate base, e.g. pyridine, DMAP, NMM, TEA, NaHCO₃, 2,4,6-collidine or DIPEA, and a suitable reagent, e.g. oxalyl chloride, cyanuric fluoride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (F) may be carried out using known procedures for preparation of amides from carboxylic acids, or analogously, e.g. as hereinafter described in the Examples. It may be carried out in a solvent, e.g. DCM, MeCN, H₂O, EtOAc or DMF, in the presence of an appropriate base, e.g. pyridine, DMAP, NMM, TEA, NaHCO₃, 2,4,6-collidine or DIPEA, and a suitable reagent, e.g. oxalyl chloride, cyanuric fluoride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (G) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (H) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (I) may be carried out using known procedures for preparation of lactones, or analogously, e.g. as hereinafter described in the Examples. It may be carried out in an organic solvent, e.g. CHCl₃, benzene, toluene, EtOH or THF, in the presence of a suitable reagent, e.g. TsOH, MsOH, NaOH, pivaloyl chloride/TEA or DMAP/BOP. The reaction temperature may be from 0° C. to 100° C., or at the reflux temperature of the solvent if <100° C., but conveniently room temperature.

Process (J) may be carried out using known procedures for preparation of lactones, or analogously, e.g. as hereinafter described in the Examples. It may be carried out in an organic solvent, e.g. CHCl₃, benzene, toluene, EtOH or THF, in the presence of a suitable reagent, e.g. TsOH, MsOH, NaOH, pivaloyl chloride/TEA or DMAP/BOP. The reaction temperature may be from 0° C. to 100° C., or at the reflux temperature of the solvent if <100° C., but conveniently room temperature.

Process (K) may be carried out using known procedures for preparation of amides from lactones, or analogously, e.g. as hereinafter described in the Examples. It may be carried out in an organic solvent, e.g. DCM, THF or MeOH, in the presence of a suitable reagent, e.g. TEA. The reaction temperature may be from 0° C. to 100° C., or at the reflux temperature of the solvent if <100° C., but conveniently room temperature.

Process (L) may be carried out using known procedures for preparation of amides from carboxylic acids, or analogously, e.g. as hereinafter described in the Examples. It may be carried out in an organic solvent, e.g. DCM, MeCN, EtOAc or DMF, in the presence of an appropriate base, e.g. pyridine, DMAP, NMM, TEA, 2,4,6-collidine or DIPEA, and a suitable reagent, e.g. oxalyl chloride, EDC/HOBt, DCC/HOBt, HBTU, HATU, PyBOP or TBTU. The reaction temperature may be from 0° C. to 100° C., or at the reflux temperature of the solvent if <100° C., but conveniently room temperature.

Process (M) may be carried out using known procedures for preparation of alcohols from ketones, or analogously, e.g. as hereinafter described in the Examples. It may be carried out in an organic solvent, e.g. THF, in the presence of a suitable reagent, e.g. NaBH₄, Zn(BH₄)₂, Ph₂SiH₂ in the presence of a suitable catalyst, e.g. Rh(PPh₃)₃Cl or Rh(I)-2-(2-pyridyl)-4-carbomethoxy-1,3-thiazolidine, or, alternatively, in the presence of H₂ and a suitable catalyst, e.g. Ru/C, Rh-DIOP or Rh—CYDIOP. The reaction temperature may be from 0° C. to 100° C., or at the reflux temperature of the solvent if <100° C., but conveniently room temperature.

Compounds of formula (II) are either commercially available or may be prepared by known methods (e.g. Bioorg. Med. Chem. Lett. 1998, 8, 2123; J. Am. Chem. Soc. 1971, 93, 3471; Tetrahedron: Asymmetry 1996, 7, 1267; Tetrahedron: Asymmetry 2006, 17, 252; J. Org. Chem. 2004, 69, 8565).

Compounds of formula (III) are either commercially available or may be prepared by known methods (e.g. J. Med. Chem. 2004, 47, 2995).

Compounds of formula (V), (VI), (VII) and (XII) are either commercially available or may be prepared by known methods.

The protection and deprotection of functional groups is described in ‘Protective Groups in Organic Synthesis’, 2^{nd} Ed, T. W. Greene and P. G. M. Wuts, Wiley-Interscience (1991) and ‘Protecting Groups’, P. J. Kocienski, Georg Thieme Verlag (1994).

A further embodiment of the invention encompasses pharmaceutically acceptable salts of the compounds of formula (I). Where the compound is sufficiently acidic, pharmaceutically-acceptable salts include, but are not limited to, an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Where the compound is sufficiently basic, pharmaceutically acceptable salts include, but are not limited to, acid addition salts such as a hydrochloride, hydrobromide, phosphate, acetate, fumarate, maleate, tartrate, citrate, oxalate, methanesulfonate or p-toluenesulfonate salt.

There may be more than one cation or anion depending on the number of charged functions and the valency of the cations or anions.

The compounds of formula (I) have chiral centers and some have geometric isomeric centers (E- and Z-isomers), and it is understood that the invention encompasses all such optical, diastereoisomeric and geometric isomers.

Medical and Pharmaceutical Use

The compounds of the invention are thus expected to be useful in those conditions where inhibition of thrombin is beneficial (as determined by reference to a clinically relevant end-point, e.g. conditions, such as thrombo-embolisms, where inhibition of thrombin is required or desired, and/or conditions where anticoagulant therapy is indicated), including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases are usually designated as thrombophilia conditions. These conditions include, but are not limited to, inherited or acquired activated protein C resistance, such as the factor V-mutation (factor V Leiden), inherited or acquired deficiencies in antithrombin III, protein C, protein S, heparin cofactor II, and conditions with increased plasma levels of the coagulation factors such as caused by the prothrombin G20210A mutation. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (e.g. disseminated intravascular coagulation (DIC)) and vascular injury in general (e.g. due to trauma or surgery). Furthermore, low physical activity, low cardiac output or high age are known to increase the risk of thrombosis and hypercoagulability may be just one of several factors underlying the increased risk. These conditions include, but are not limited to, prolonged bed rest, prolonged air travelling, hospitalization for an acute medical disorder such as cardiac insufficiency or respiratory insufficiency. Further conditions with increased risk of thrombosis with hypercoagulability as one component are pregnancy and hormone treatment (e.g. oestrogen).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (e.g. deep venous thrombosis, DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (e.g. non-valvular or valvular atrial fibrillation) or from the left ventricle after transmural myocardial infarction, or caused by congestive heart failure; prophylaxis of re-occlusion (i.e. thrombosis) after thrombolysis, percutaneous trans-luminal interventions (PTI) and coronary bypass operations; the prevention of thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, chronic obstructive pulmonary disease, septic shock, septicaemia, inflammatory responses, which include, but are not limited to, edema, acute or chronic atherosclerosis such as coronary arterial disease and the formation of atherosclerotic plaques, cardiac insufficiency, cerebral arterial disease, cerebral infarction, cerebral thrombosis, cerebral embolism, peripheral arterial disease, ischemia, angina (including unstable angina), reperfusion damage, restenosis after percutaneous trans-luminal interventions (PTI) and coronary artery bypass surgery.

Compounds of the invention that inhibit trypsin and/or thrombin may also be useful in the treatment of pancreatitis.

The compounds of the invention are thus indicated both in the therapeutic and/or prophylactic treatment of these conditions.

The compounds of the invention have the advantage that they may be more efficacious, be less toxic, be more selective (e.g. for inhibiting thrombin over other serine proteases, in particular trypsin and those involved in haemostasis), be more potent, produce fewer side effects, be more easily absorbed, and/or have a better pharmacokinetic profile (e.g. higher oral bioavailability and/or lower clearance), than compounds known in the prior art.

Pharmaceutical Formulation

According to a further aspect of the present invention, there is provided a method of treatment of a condition where inhibition of thrombin is required which method comprises administration of a therapeutically effective amount of a compound of the invention to a person suffering from, or susceptible to, such a condition.

The compounds of the invention will normally be administered orally, intravenously, subcutaneously, buccally, rectally, dermally, nasally, tracheally, bronchially, by any other parenteral route or via inhalation, in the form of pharmaceutical preparations comprising a compound of the invention either as a free base, or a pharmaceutically acceptable non-toxic organic or inorganic acid addition salt, in a pharmaceutically acceptable dosage form.

Preferred route of administration of compounds of the invention is oral.

Depending upon the disorder and patient to be treated and the route of administration, the compositions may be administered at varying doses.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than thrombin (e.g. synthetic FXa, FVIIa, FIXa and FXIa inhibitors, and rNAPc2), the antiplatelet agents acetylsalicylic acid and dipyridamole), thromboxane receptor and/or synthetase inhibitors, fibrinogen receptor antagonists, prostacyclin mimetics, phosphodiesterase inhibitors, ADP-receptor (P2X₁, P2Y₁, P2Y₁₂ [e.g. ticlopidine, clopidogrel, cangrelor, satigrel and AZD6140]) antagonists, inhibitors of phosphoinositide 3-kinase beta or gamma, inhibitors of carboxypeptidase U (CPU or TAFIa) and inhibitors of plasminogen activator inhibitor-1 (PAI-1, e.g. SCH530348 and E-5555).

The compounds of the invention may further be combined and/or co-administered with thrombolytics such as one or more of tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

According to a further aspect of the invention there is thus provided a pharmaceutical formulation including a compound of the invention, in admixture with a pharmaceutically acceptable adjuvant, diluent or carrier.

Suitable daily doses of the compounds of the invention in therapeutic treatment of humans are about 0.001-100 mg/kg body weight at peroral administration and 0.001-50 mg/kg body weight at parenteral administration.

For the avoidance of doubt, as used herein, the term “treatment” includes therapeutic and/or prophylactic treatment.

### EXAMPLES

The invention will now be further explained by reference to the following examples. In the examples, high resolution mass spectra were recorded on a Micromass LCT mass spectrometer equipped with an electrospray interface (LC-HRMS). ¹H NMR measurements were performed on Varian UNITY plus 400, 500 and 600 spectrometers, operating at ¹H frequencies of 400, 500 and 600 MHz respectively. Chemical shifts are given in ppm with the solvent as internal standard. Flash chromatography separations were performed using Merck Silica gel 60 (0.063-0.200 mm). The compounds named below were named using AutoNom 2000 available from MDL Information Systems GmbH.

The following abbreviations are used:
DMF Dimethylformamide
HATU O-(7-Azobenzotriazol-1-yl)-1,1,3,3-tetramethyluronium hexafluorophosphate
PyBOP Benzotriazol-1-yloxytripyrrolidinophosphonium hexafluorophosphate
TBTU O-Benzotriazolyl tetramethylisouronium tetrafluoroborate
EDC 1-(3-Dimethylaminopropyl)-3-ethylcarbodiimide
DMAP 4-(Dimethylamino)pyridine
NMM N-Methylmorpholine
TEA Triethylamine
DCM Dichloromethane
DCC Dicyclohexylcarbodiimide
BOP Benzotriazol-1-yloxytris(dimethylamino)phosphonium hexafluorophosphate
HBTU O-(Benzotriazol-1-yl)-N,N,N′,N′-tetramethyluronium hexafluorophosphate;
HATU O-(7-Azabenzotriazol-1-yl)-N,N,N′,N′-tetramethyluronium hexafluorophosphate;
HOBt 1-Hydroxybenzotriazole;
HOAT 1-Hydroxy-7-azabenzotriazole;
DIPEA N,N-Diisopropylethylamine;
DIOP Phosphine, [(2,2-dimethyl-1,3-dioxolane-4,5-diyl)bis(methylene)]bis[diphenyl-, trans-
CYDIOP Phosphine, [(2,2-dimethyl-1,3-dioxolane-4,5-diyl)bis(methylene)]bis[dicyclohexyl-, trans-
NMP 1-N-Methyl-2-pyrrolidinone;
TBME tert-Butyl methyl ether

Preparation

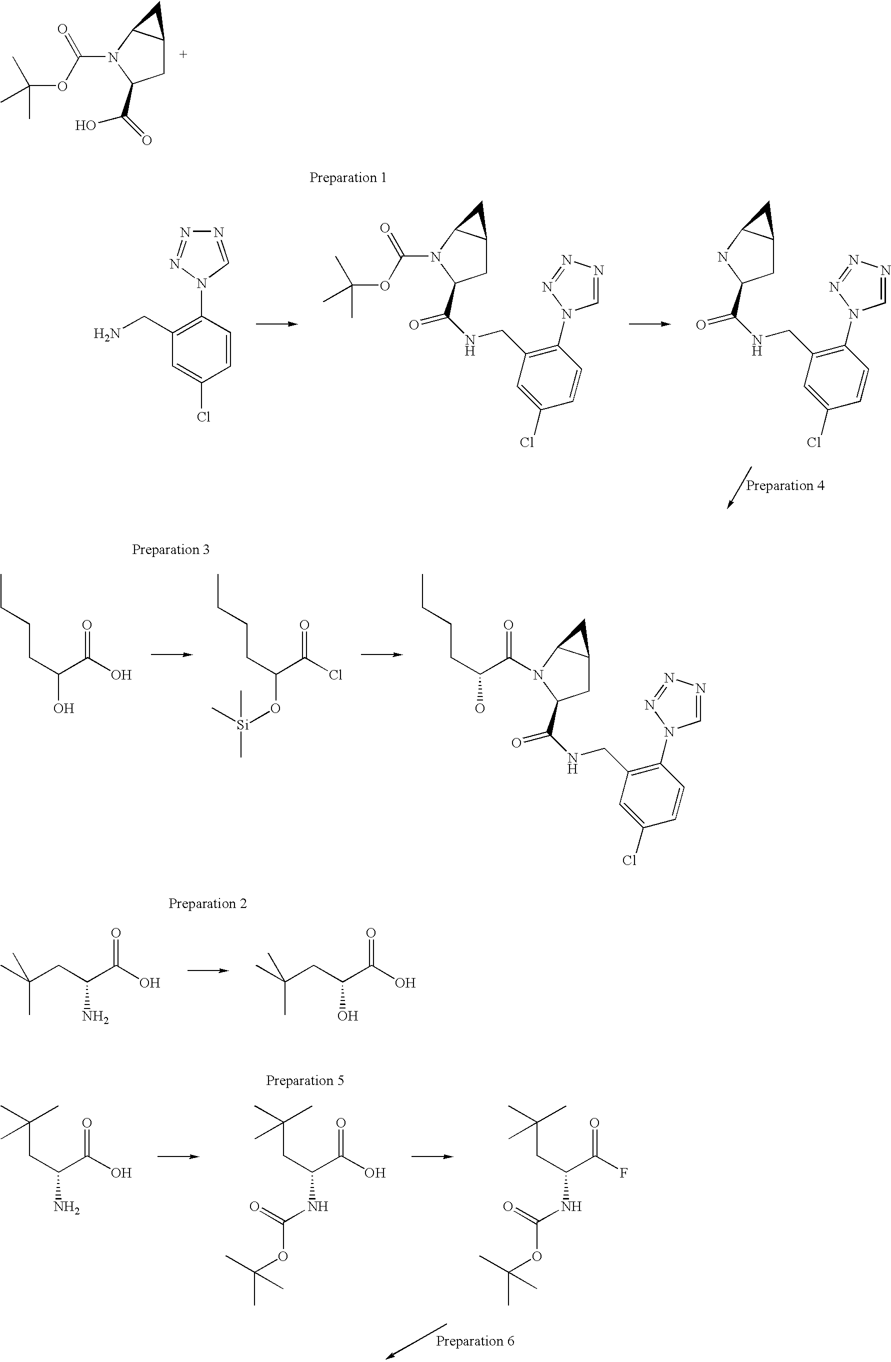

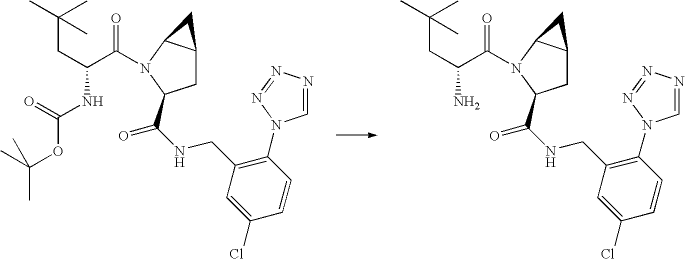

Preparation 1

### (1S,3S,5S)-2-Aza-bicyclo[3.1.0]hexane-3-carboxylic acid 5-chloro-2-tetrazol-1-yl-benzylamide

### a) (1S,3S,5S)-3-(5-Chloro-2-tetrazol-1-yl-benzylcarbamoyl)-2-aza-bicyclo[3.1.0]hexane-2-carboxylic acid tert-butyl ester

To a solution of (1S,3S,5S)-2-aza-bicyclo[3.1.0]hexane-2,3-dicarboxylic acid 2-tert-butyl ester (1.000 g, 4.400 mmol) and 5-chloro-2-tetrazol-1-yl-benzylamine (1.015 g, 4.840 mmol) in DCM (35 mL) was added HOBt (1.011 g, 6.600 mmol), EDC (1.265 g, 6.600 mmol) and TEA (1.22 mL, 8.80 mmol). The solution was stirred at room temperature overnight. The mixture was diluted with DCM and washed with water, 1M aqueous HCl and saturated aqueous NaHCO₃. The organic phase was dried, filtered and concentrated under reduced pressure. Purification using flash chromatography (heptane/EtOAc, 9/1→0/1) gave the subtitle product (1.834 g, 100%).

### b) (1S,3S,5S)-2-Aza-bicyclo[3.1.0]hexane-3-carboxylic acid 5-chloro-2-tetrazol-1-yl-benzylamide

To a solution of (1S,3S,5S)-3-(5-chloro-2-tetrazol-1-yl-benzylcarbamoyl)-2-aza-bicyclo[3.1.0]hexane-2-carboxylic acid tert-butyl ester (1.834 g, 4.378 mmol) in MeOH (30 mL) was added concentrated aqueous HCl (15 mL). The reaction was stirred at room temperature for 1 hour. Concentration under reduced pressure gave the title compound (1.36 g, 87%) as the HCl-salt.

Preparation 2

### (R)-2-Hydroxy-4,4-dimethyl-pentanoic acid

A solution of sodium nitrite (0.801 g, 11.600 mmol) in water (3.75 mL) was added dropwise to a stirred solution of (R)-2-amino-4,4-dimethyl-pentanoic acid (0.843 g, 5.806 mmol) in aqueous sulfuric acid (12 mL, 0.5 M, 6 mmol) at −10° C. The reaction mixture was allowed to slowly attain room temperature overnight. Sodium chloride (1.5 g) was added and the solution was extracted with TBME (4×15 mL). The combined organic extracts were dried over anhydrous magnesium sulfate, filtered and concentrated under reduced pressure to give the title compound (0.455 g, 54%).

Preparation 3

### 2-Trimethylsilanyloxy-hexanoyl chloride

TMSCl (1.769 mL, 14.000 mmol) was added dropwise to a solution of 2-hydroxy-hexanoic acid (0.925 g, 7.000 mmol), DMAP (0.017 g, 0.140 mmol) and pyridine (1.189 mL, 14.700 mmol) in DCM (14 mL) at room temperature. The mixture was stirred at room temperature for 4 hours. The reaction mixture was cooled to 0° C. and a few drops of DMF were added, followed by dropwise addition of oxalyl chloride (2 M in DCM, 3.5 mL, 7 mmol). The mixture was stirred for 1 hour at 0° C. and the reaction mixture was then allowed to attain room temperature. The resulting solution was used directly in the next reaction step assuming quantitative formation of 2-trimethylsilanyloxy-hexanoyl chloride.

Preparation 4

### (1S,3S,5S)-2-((R)-2-Hydroxy-hexanoyl)-2-aza-bicyclo[3.1.0]hexane-3-carboxylic acid 5-chloro-2-tetrazol-1-yl-benzylamide

A solution of 2-trimethylsilanyloxy-hexanoyl chloride (see Preparation 3) in DCM (2.1 mL, 0.3 M, 0.63 mmol) was added to a solution of (1S,3S,5S)-2-aza-bicyclo[3.1.0]hexane-3-carboxylic acid 5-chloro-2-tetrazol-1-yl-benzylamide (0.255 g, 0.633 mmol) (see Preparation 1) and pyridine (0.51 mL, 6.3 mmol) in DCM (4 mL). The mixture was stirred at room temperature for 3 days. TFA (0.732 mL, 9.5 mmol) and a few drops of MeOH was then added and the reaction mixture was stirred for additional 15 min. The mixture was diluted with DCM, washed with 1M aqueous HCl and saturated aqueous NaHCO₃, dried through a phase separator and concentrated under reduced pressure. Flash chromatography (heptane/EtOAc, 5/2→0/1) gave a diastereomeric mixture of (1S,3S,5S)-2-(2-hydroxy-hexanoyl)-2-aza-bicyclo[3.1.0]hexane-3-carboxylic acid 5-chloro-2-tetrazol-1-yl-benzylamide (0.1 58 g, 58%). The enantiomers were separated using chiral chromatography (Chiralpak IA, 250×20 mm, 5 μm, heptane/EtOH/TEA, 30/70/0.1, flow 15 mL/min, 40° C., concentration 50 mg/mL) to give the title compound [α]²⁰_{D}+20.1 (c 1.0, MeCN), 98.7% ee. HRMS (ESI) calculated for C₂₀H₂₅ClN₆O₃ 433.1755, found 433.1754.

Preparation 5

### ((R)-1-Fluorocarbonyl-3,3-dimethyl-butyl)-carbamic acid tert-butyl ester

### a) (R)-2-tert-Butoxycarbonylamino-4,4-dimethyl-pentanoic acid

To a solution of (R)-2-amino-4,4-dimethyl-pentanoic acid (1.452 g, 10 mmol) in water (10 mL) was added NaOH (0.44 g, 11 mmol) and a solution of Boc-anhydride (2.292 g, 10.5 mmol) in THF (10 mL). The cloudy mixture, which gradually became clear and then cloudy again, was stirred at room temperature over night. Most of the THF was evaporated and the residue was acidified with 1M NaHSO₄ and extracted (3×) with DCM. The combined organic phases were dried, filtered and evaporated to give the pure product (2.44 g, 99.5%).

### b) ((R)-1-Fluorocarbonyl-3,3-dimethyl-butyl)-carbamic acid tert-butyl ester

To a solution of (R)-2-tert-butoxycarbonylamino-4,4-dimethyl-pentanoic acid (1.23 g, 5 mmol) in DCM (25 mL) and pyridine (0.791 g, 10 mmol) was added cyanuric fluoride (1.35 g, 10 mmol) at −10° C. After 2 h at this temperature, the mixture was diluted with DCM and quenched with saturated NaHCO₃. The organic phase was washed with water, dried, filtered and evaporated to give the crude product as an almost colorless solid (1.22 g, 99%) which was used immediately in the next reaction.

Preparation 6

### a) {(R)-1-[(1S,3S,5S)-3-(5-Chloro-2-tetrazol-1-yl-benzylcarbamoyl)-2-aza-bicyclo[3.1.0]hexane-2-carbonyl]-3,3-dimethyl-butyl}-carbamic acid tert-butyl ester

A solution of ((R)-1-fluorocarbonyl-3,3-dimethyl-butyl)-carbamic acid tert-butyl ester (see Preparation 5) in DCM (6 mL) (0.148 g, 0.60 mmol) was added to a suspension of (1S,3S,5S)-2-aza-bicyclo[3.1.0]hexane-3-carboxylic acid 5-chloro-2-tetrazol-1-yl-benzylamide (0.107 g, 0.30 mmol) (see Preparation 1) and NaHCO₃ in water (6 mL) to give a two-phase mixture. A small amount of DMF was added to help solubilize the ingredients and the mixture was vigorously stirred at room temperature overnight. The mixture was diluted with DCM, the phases were separated and the organic phase was washed with 1M HCl and sat. NaHCO₃, dried through a phase separator and evaporated. Purification using HPLC (Preparative conditions: Kromasil C8, 300×50.8 mm, 10 μm, gradient 65-85% MeCN in aq. NH₄OAc buffer during 20 min, flow 60 mL/min) gave the title compound (0.150 g, 92%).

### b) (1S,3S,5S)-2-((R)-2-Amino-4,4-dimethyl-pentanoyl)-2-aza-bicyclo[3.1.0]hexane-3-carboxylic acid 5-chloro-2-tetrazol-1-yl-benzylamide

{(R)-1-[(1S,3S,5S)-3-(5-Chloro-2-tetrazol-1-yl-benzylcarbamoyl)-2-aza-bicyclo[3.1.0]hexane-2-carbonyl]-3,3-dimethyl-butyl}-carbamic acid tert-butyl ester (01.50 g, 0274 mmol) was dissolved in MeOH (2 mL) and conc. HCl (2 mL) was added. Some gas evolution occurred, stirred at room temperature for 2 hour and then evaporated to give the title compound (0.115 g, 87%) as the HCl-salt.

The following examples were synthesized according to the procedures described above using the appropriate starting materials.

### Example 1

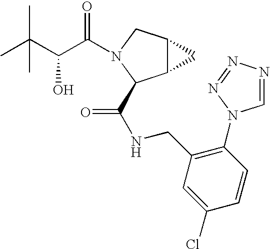

### (1S,2S,5R)-3-((R)-2-Hydroxy-3,3-dimethyl-butyryl)-3-aza-bicyclo[3.1.0]hexane-2-carboxylic acid 5-chloro-2-tetrazol-1-yl-benzylamide

¹H NMR (500 MHz, CDCl₃): δ 9.07 (s, 1H), 7.60 (d, 1H), 7.43 (t, 1H), 7.41 (dd, 1H), 7.25 (d, 1H), 4.50 (s, 1H), 4.25 (dd, 1H), 4.18 (dd, 1H), 3.91 (d, 1H), 3.79 (d, 1H), 3.75 (dd, 1H), 3.08 (d, 1H), 1.75 (m, 1H), 1.66 (m, 1H), 0.94 (s, 9H), 0.75 (m, 1H), 0.21 (m, 1H). HRMS (ESI) calculated for C₂₀H₂₅ClN₆O₃ 433.1755 (M+H)⁺, found 433.1757.

### Example 2

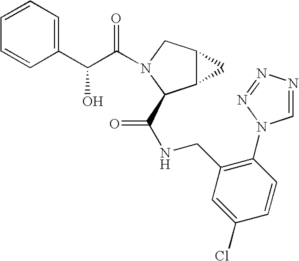

### (1S,2S,5R)-3-((R)-2-Hydroxy-2-phenyl-acetyl)-3-aza-bicyclo[3.1.0]hexane-2-carboxylic acid 5-chloro-2-tetrazol-1-yl-benzylamide

¹H NMR (400 MHz, CD₃CN) for the major rotamer: δ 9.22 (s, 1H), 7.66 (d, 1H), 7.52 (dd, 1H), 7.43 (d, 1H), 7.40-7.19 (m, 6H), 5.08 (s, 1H), 4.41 (s, 1H), 4.21 (m, 2H), 3.59 (dd, 1H), 3.31 (d, 1H), 1.49 (m, 2H), 0.53 (m, 1H), −0.41 (m, 1H). HRMS (ESI) calculated for C₂₂H₂₁ClN₆O₃ 453.1442 (M+H)⁺, found 453.1440.

### Example 3

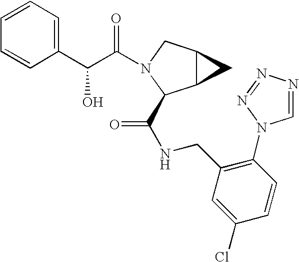

### (1R,2S,5S)-3-((R)-2-Hydroxy-2-phenyl-acetyl)-3-aza-bicyclo[3.1.0]hexane-2-carboxylic acid 5-chloro-2-tetrazol-1-yl-benzylamide

¹H NMR (400 MHz, CD₃CN): δ 9.23 (s, 1H), 7.80 (d, 1H), 7.52 (dd, 1H), 7.44-7.32 (m, 6H), 6.97 (m, 1H), 5.04 (d, 1H), 4.34 (d, 1H), 4.19 (m, 2H), 3.60 (d, 1H), 3.04 (m, 1H), 1.79 (m, 1H), 1.55 (m, 1H), 0.64 (m, 2H). HRMS (ESI) calculated for C₂₂H₂₁ClN₆O₃ 453.1442 (M+H)⁺, found 453.1441.

### Example 4

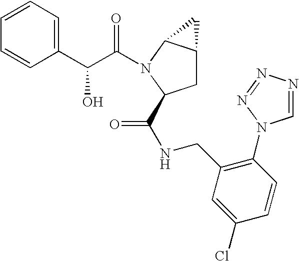

### (1R,3S,5R)-2-((R)-2-Hydroxy-2-phenyl-acetyl)-2-aza-bicyclo[3.1.0]hexane-3-carboxylic acid 5-chloro-2-tetrazol-1-yl-benzylamide

¹H NMR (400 MHz, CDCl₃): δ 9.10 (s, 1H), 7.59 (d, 1H), 7.55 (m, 1H), 7.36 (dd, 1H), 7.26-7.21 (m, 6H), 5.30 (s, 1H), 4.29 (dd, 1H), 4.17 (d, 2H), 3.21 (m, 1H), 2.33 (m, 1H), 1.84 (m, 1H), 1.68 (m, 1H), 0.39 (m, 1H), −0.49 (m, 1H). HRMS (ESI) calculated for C₂₂H₂₁ClN₆O₃ 453.1442 (M+H)⁺, found 453.1474.

### Example 5

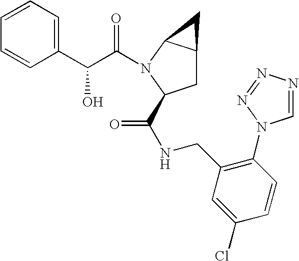

### (1S,3S,5S)-2-((R)-2-Hydroxy-2-phenyl-acetyl)-2-aza-bicyclo[3.1.0]hexane-3-carboxylic acid 5-chloro-2-tetrazol-1-yl-benzylamide

¹H NMR (400 MHz, CDCl₃): δ 9.10 (s, 1H), 7.65 (d, 1H), 7.59 (m, 1H), 7.42 (dd, 1H), 7.35 (m, 5H), 7.26 (d, 1H), 5.31 (s, 1H), 4.73 (dd, 1H), 4.19 (ddd, 2H), 3.12 (m, 1H), 2.24 (m, 2H), 1.49 (m, 1H), 0.96 (m, 1H), 0.74 (m, 1H). HRMS (ESI) calculated for C₂₂H₂₁ClN₆O₃ 453.1442 (M+H)⁺, found 453.1475.

### Example 6

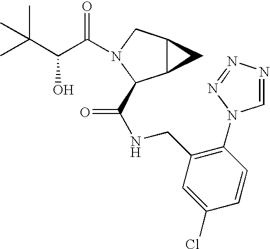

### (1R,2S,5S)-3-((R)-2-Hydroxy-3,3-dimethyl-butyryl)-3-aza-bicyclo[3.1.0]hexane-2-carboxylic acid 5-chloro-2-tetrazol-1-yl-benzylamide

¹H NMR (400 MHz, CD₃CN): δ 9.22 (s, 1H), 7.80 (d, 1H), 7.49 (dd, 1H), 7.41 (d, 1H), 6.92 (m, 1H), 4.35 (d, 1H), 4.15 (d, 2H), 3.84 (m, 2H), 3.76 (d, 1H), 1.84 (m, 1H), 1.72 (m, 1H), 0.94 (s, 9H), 0.71 (m, 1H), 0.66 (m, 1H). HRMS (ESI) calculated for C₂₀H₂₅ClN₆O₃ 433.1755 (M+H)⁺, found 433.1754.

### Example 7

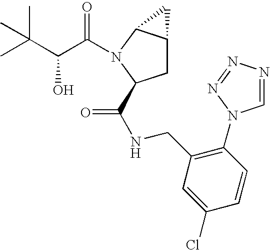

### (1R,3S,5R)-2-((R)-2-Hydroxy-3,3-dimethyl-butyryl)-2-aza-bicyclo[3.1.0]hexane-3-carboxylic acid 5-chloro-2-tetrazol-1-yl-benzylamide

¹H NMR (400 MHz, CD₃CN): δ 9.22 (s, 1H), 7.71 (d, 1H), 7.50 (dd, 1H), 7.41 (d, 1H), 7.06 (m, 1H), 4.20 (s, 1H), 4.14 (m, 3H), 3.61 (m, 1H), 2.18 (m, 2H), 1.81 (m, 1H), 0.98 (s, 9H), 0.94 (m, 1H), 0.53 (m, 1H). HRMS (ESI) calculated for C₂₀H₂₅ClN₆O₃ 433.1755 (M+H)⁺, found 433.1760.

### Example 8

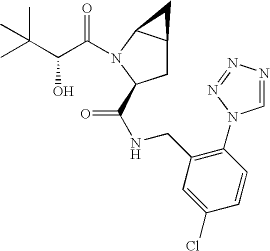

### (1S,3S,5S)-2-((R)-2-Hydroxy-3,3-dimethyl-butyryl)-2-aza-bicyclo[3.1.0]hexane-3-carboxylic acid 5-chloro-2-tetrazol-1-yl-benzylamide

¹H NMR (400 MHz, CD₃CN): δ 9.20 (s, 1H), 7.68 (d, 1H), 7.50 (dd, 1H), 7.41 (d, 1H), 7.28 (m, 1H), 4.64 (dd, 1H), 4.20 (m, 2H), 4.05 (dd, 1H), 3.70 (m, 1H), 2.37 (m, 1H), 2.01 (dd, 1H), 1.66 (m, 1H), 1.13 (m, 1H), 0.99 (s, 9H), 0.69 (m, 1H). HRMS (ESI) calculated for C₂₀H₂₅ClN₆O₃ 433.1755 (M+H)⁺, found 433.1751.

### Example 9

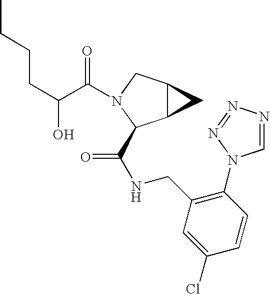

### (1R,2S,5S)-3-(2-Hydroxy-hexanoyl)-3-aza-bicyclo[3.1.0]hexane-2-carboxylic acid 5-chloro-2-tetrazol-1-yl-benzylamide

¹H NMR (400 MHz, CD₃CN) for the major rotamer: δ 9.21 (s, 1H), 7.64 (d, 1H), 7.50 (dd, 1H), 7.41 (d, 1H), 7.17 (m, 1H), 4.34 (s, 1H), 4.24-4.12 (m, 3H), 3.69 (dd, 1H), 3.62 (d, 1H), 1.65-1.54 (m, 3H), 1.41-1.28 (m, 5H), 0.89 (m, 3H), 0.77 (m, 1H), 0.15 (m, 1H). HRMS (ESI) calculated for C₂₀H₂₅ClN₆O₃ 433.1755 (M+H)⁺, found 433.1751.

### Example 10

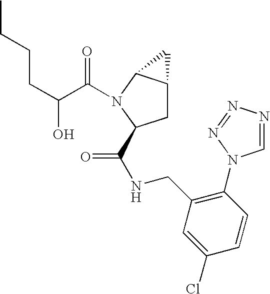

### (1R,3S,5R)-2-(2-Hydroxy-hexanoyl)-2-aza-bicyclo[3.1.0]hexane-3-carboxylic acid 5-chloro-2-tetrazol-1-yl-benzylamide

¹H NMR (400 MHz, CD₃CN): δ 9.21 (s, 1H), 7.68 (d, 1H), 7.49 (dd, 1H), 7.41 (d, 1H), 7.12 (m, 1H), 4.46 (dd, 1H), 4.14 (m, 3H), 3.44 (m, 1H), 2.26-2.09 (m, 2H), 1.82 (m, 2H), 1.57 (m, 1H), 1.40-1.31 (m, 4H), 0.96-0.89 (m, 4H), 0.52 (m, 1H). HRMS (ESI) calculated for C₂₀H₂₅ClN₆O₃ 433.1755 (M+H)⁺, found 433.1735.

### Example 11

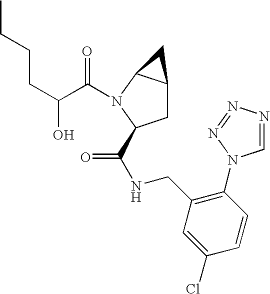

### (1S,3S,5S)-2-(2-Hydroxy-hexanoyl)-2-aza-bicyclo[3.1.0]hexane-3-carboxylic acid 5-chloro-2-tetrazol-1-yl-benzylamide

¹H NMR (400 MHz, CD₃CN): δ 9.19 (s, 1H), 7.68 (d, 1H), 7.50 (dd, 1H), 7.41 (d, 1H), 7.17 (m, 1H), 4.60 (dd, 1H), 4.34 (m, 1H), 4.18 (dd, 1H), 4.07 (dd, 1H), 3.45 (m, 1H), 2.41 (m, 1H), 1.79-1.63 (m, 2H), 1.57-1.29 (m, 6H), 0.97-0.89 (m, 4H), 0.74 (m, 1H). HRMS (ESI) calculated for C₂₀H₂₅ClN₆O₃ 433.1755 (M+H)⁺, found 433.1754.

### Example 12

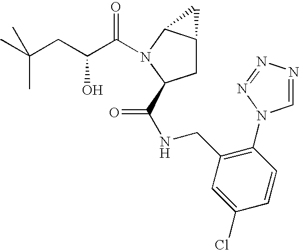

### (1R,3S,5R)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide

¹H NMR (400 MHz, CD₃CN): δ 9.20 (s, 1H), 7.69 (d, 1H), 7.51 (dd, 1H), 7.41 (d, 1H), 7.04 (m, 1H), 4.59 (t, 1H), 4.15 (m, 3H), 3.41 (m, 1H), 3.33 (d, 1H), 2.20 (m, 1H), 1.81 (m, 2H), 1.26 (dd, 1H), 1.03 (s, 9H), 0.97 (m, 1H), 0.55 (m, 1H). HRMS (ESI) calculated for C₂₁H₂₇ClN₆O₃ 447.1911 (M+H)⁺, found 447.1884.

### Example 13

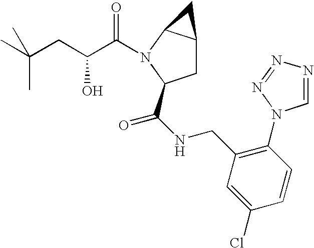

### (1S,3S,5S)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide

¹H NMR (400 MHz, CD₃CN): δ 9.19 (s, 1H), 7.69 (d, 1H), 7.51 (dd, 1H), 7.41 (d, 1H), 7.12 (m, 1H), 4.59 (dd, 1H), 4.48 (d, 1H), 4.19 (dd, 1H), 4.06 (dd, 1H), 3.38 (m, 1H), 2.42 (m, 1H), 1.96 (m, 1H), 1.66 (m, 1H), 1.62 (d, 1H), 1.42 (dd, 1H), 1.03 (s, 9H), 0.98 (m, 1H), 0.77 (m, 1H). HRMS (ESI) calculated for C₂₁H₂₇ClN₆O₃ 447.1911 (M+H)⁺, found 447.1870.

### Example 14

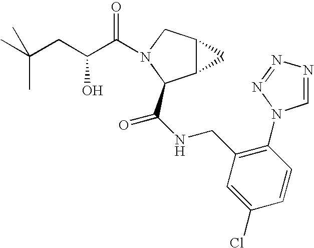

### (1S,2S,5R)-3-((R)-2-Hydroxy-4,4-dimethyl-pentanoyl)-3-aza-bicyclo[3.1.0]hexane-2-carboxylic acid 5-chloro-2-tetrazol-1-yl-benzylamide

¹H NMR (400 MHz, CD₃CN) for the major rotamer: δ 9.19 (s, 1H), 7.64 (d, 1H), 7.50 (dd, 1H), 7.41 (d, 1H), 7.14 (m, 1H), 4.32 (s, 1H), 4.20 (m, 3H), 3.70 (dd, 1H), 3.58 (d, 1H), 1.65 (m, 1H), 1.56 (m, 1H), 1.39 (d, 1H), 1.29 (dd, 1H), 0.99 (s, 9H), 0.77 (m, 1H), 0.18 (m, 1H). HRMS (ESI) calculated for C₂₁H₂₇ClN₆O₃ 447.1911 (M+H)⁺, found 447.1878.

### Example 15

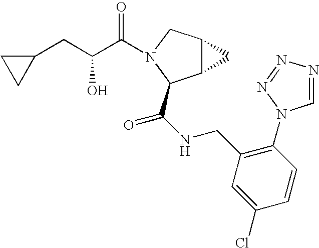

### (1S,2S,5R)-3-((R)-3-Cyclopropyl-2-hydroxy-propionyl)-3-aza-bicyclo[3.1.0]hexane-2-carboxylic acid 5-chloro-2-tetrazol-1-yl-benzylamide

¹H NMR (500 MHz, CDCl₃): δ 9.00 (s, 1H), 7.60 (d, 1H), 7.45 (dd, 1H), 7.27 (m, 1H), 7.20 (m, 1H), 4.50 (s, 1H), 4.26 (m, 3H), 3.68 (dd, 1H), 3.59 (d, 1H), 1.86 (m, 1H), 1.70 (m, 1H), 1.59 (m, 1H), 1.36 (m, 1H), 0.83 (m, 2H), 0.51 (m, 2H), 0.10 (m, 3H). HRMS (ESI) calculated for C₂₀H₂₃ClN₆O₃ 431.1598 (M+H)⁺, found 431.1628.

### Example 16

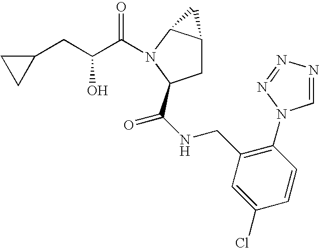

### (1R,3S,5R)-2-((R)-3-Cyclopropyl-2-hydroxy-propionyl)-2-aza-bicyclo[3.1.0]hexane-3-carboxylic acid 5-chloro-2-tetrazol-1-yl-benzylamide

¹H NMR (500 MHz, CDCl₃): δ 9.00 (s, 1H), 7.61 (d, 1H), 7.45 (dd, 1H), 7.25 (m, 2H), 4.59 (dd, 1H), 4.44 (dd, 1H), 4.25 (m, 2H), 3.33 (m, 1H), 2.69 (m, 1H), 1.95 (m, 2H), 1.76 (m, 1H), 1.51 (m, 1H), 1.08 (m, 1H), 0.92 (m, 1H), 0.50 (m, 3H), 0.10 (m, 2H). HRMS (ESI) calculated for C₂₀H₂₃ClN₆O₃ 431.1598 (M+H)⁺, found 431.1587.

### Example 17

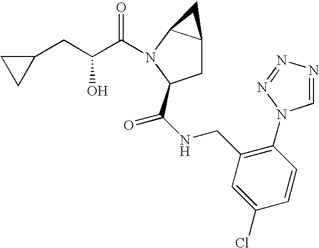

### (1S,3S,5S)-2-((R)-3-Cyclopropyl-2-hydroxy-propionyl)-2-aza-bicyclo[3.1.0]hexane-3-carboxylic acid 5-chloro-2-tetrazol-1-yl-benzylamide

¹H NMR (500 MHz, CDCl₃): δ 9.00 (s, 1H), 7.60 (m, 1H), 7.57 (d, 1H), 7.44 (dd, 1H), 7.26 (d, 1H), 4.74 (dd, 1H), 4.59 (dd, 1H), 4.21 (m, 2H), 3.37 (m, 1H), 2.61 (dd, 1H), 2.17 (m, 1H), 1.72 (m, 2H), 1.53 (m, 1H), 0.91 (m, 1H), 0.76 (m, 2H), 0.53 (m, 2H), 0.11 (m, 2H). HRMS (ESI) calculated for C₂₀H₂₃ClN₆O₃ 431.1598 (M+H)⁺, found 431.1592.

### Example 18

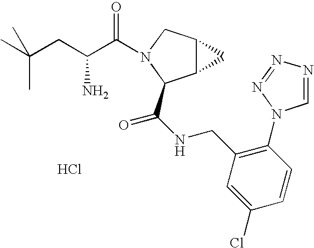

### (1S,2S,5R)-3-((R)-2-Amino-4,4-dimethyl-pentanoyl)-3-aza-bicyclo[3.1.0]hexane-2-carboxylic acid 5-chloro-2-tetrazol-1-yl-benzylamide hydrochloride

¹H NMR (400 MHz, D₂O) for the major rotamer: δ 9.55 (s, 1H), 7.71 (s, 1H), 7.65 (d, 1H), 7.53 (d, 1H), 4.44 (s, 1H), 4.36 (s, 2H), 4.23 (dd, 1H), 3.90 (dd, 1H), 3.79 (d, 1H), 1.84-1.58 (m, 4H), 1.01 (m, 10H), 0.30 (m, 1H). HRMS (ESI) calculated for C₂₁H₂₈ClN₇O₂ 446.2071 (M+H)⁺, found 446.2060.

### Example 19

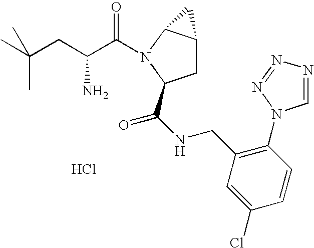

### (1R,3S,5R)-2-((R)-2-Amino-4,4-dimethyl-pentanoyl)-2-aza-bicyclo[3.1.0]hexane-3-carboxylic acid 5-chloro-2-tetrazol-1-yl-benzylamide hydrochloride

¹H NMR (400 MHz, D₂O): δ 9.55 (s, 1H), 7.71 (s, 1H), 7.64 (d, 1H), 7.53 (d, 1H), 4.63 (dd, 1H), 4.31 (m, 2H), 4.22 (dd, 1H), 3.69 (m, 1H), 2.40 (dd, 1H), 2.13-1.98 (m, 3H), 1.72 (dd, 1H), 1.14 (m, 1H), 1.04 (s, 9H), 0.80 (m, 1H). HRMS (ESI) calculated for C₂₁H₂₈ClN₇O₂ 446.2071 (M+H)⁺, found 446.2067.

### Example 20

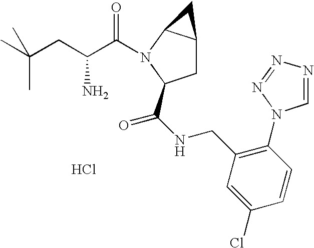

### (1S,3S,5S)-2-((R)-2-Amino-4,4-dimethyl-pentanoyl)-2-aza-bicyclo[3.1.0]hexane-3-carboxylic acid 5-chloro-2-tetrazol-1-yl-benzylamide hydrochloride

¹H NMR (400 MHz, D₂O): δ 9.54 (s, 1H), 7.69 (s, 1H), 7.64 (d, 1H), 7.52 (d, 1H), 4.71 (dd, 1H), 4.55 (m, 1H), 4.29 (dd, 2H), 3.56 (m, 1H), 2.68 (m, 1H) 2.00 (dd, 1H), 1.91-1.78 (m, 3H), 1.05 (m, 10H), 0.93 (m, 1H). HRMS (ESI) calculated for C₂₁H₂₈ClN₇O₂ 446.2071 (M+H)⁺, found 446.2061.

### Example 21

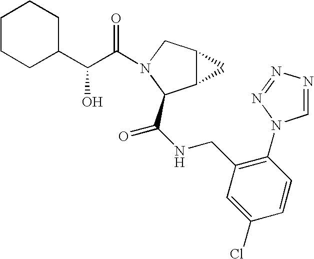

### (1S,2S,5R)-3-((R)-2-Cyclohexyl-2-hydroxy-acetyl)-3-aza-bicyclo[3.1.0]hexane-2-carboxylic acid 5-chloro-2-tetrazol-1-yl-benzylamide

¹H NMR (400 MHz, CDCl₃): δ 9.03 (s, 1H), 7.59 (d, 1H), 7.43 (dd, 1H), 7.36 (m, 1H), 7.26 (d, 1H), 4.50 (s, 1H), 4.24 (ddd, 2H), 4.01 (d, 1H), 3.66 (m, 2H), 1.83-1.13 (m, 13H), 0.82 (m, 1H), 0.10 (m, 1H). HRMS (ESI) calculated for C₂₂H₂₇ClN₆O₃ 459.1911 (M+H)⁺, found 459.1911.

### Example 22

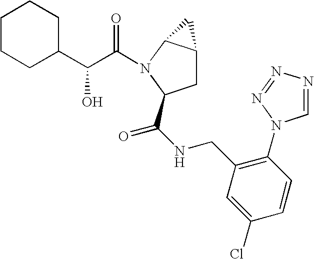

### (1R,3S,5R)-2-((R)-2-Cyclohexyl-2-hydroxy-acetyl)-2-aza-bicyclo[3.1.0]hexane-3-carboxylic acid 5-chloro-2-tetrazol-1-yl-benzylamide

¹H NMR (400 MHz, CDCl₃): δ 9.04 (s, 1H), 7.61 (d, 1H), 7.42 (dd, 1H), 7.31 (m, 1H), 7.25 (d, 1H), 4.42 (dd, 1H), 4.31 (d, 1H), 4.22 (d, 2H), 3.35 (m, 1H), 2.58 (m, 1H), 2.05-1.08 (m, 14H), 0.50 (m, 1H). HRMS (ESI) calculated for C₂₂H₂₇ClN₆O₃ 459.1911 (M+H)⁺, found 459.1923.

### Example 23

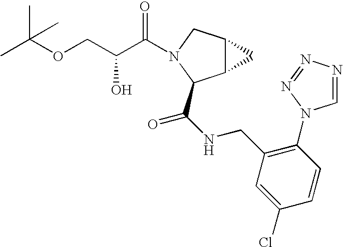

### (1S,2S,5R)-3-((R)-3-tert-Butoxy-2-hydroxy-propionyl)-3-aza-bicyclo[3.1.0]hexane-2-carboxylic acid 5-chloro-2-tetrazol-1-yl-benzylamide

¹H NMR (500 MHz, CDCl₃): δ 9.00 (s, 1H), 7.58 (d, 1H), 7.45 (dd, 1H), 7.26 (m, 2H), 4.58 (s, 1H), 4.34 (m, 1H), 4.24 (m, 2H), 4.07 (d, 1H), 3.58 (m, 2H), 3.37 (t, 1H), 1.85 (m, 1H), 1.65 (m, 1H), 1.17 (s, 9H), 0.76 (m, 1H), 0.27 (m, 1H). HRMS (ESI) calculated for C₂₁H₂₇ClN₆O4 463.1860 (M+H)⁺, found 463.1860.

### Example 24

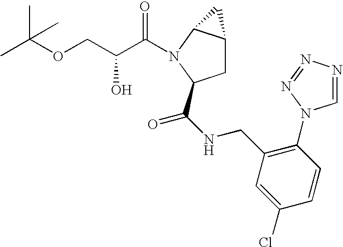

### (1R,3S,5R)-2-((R)-3-tert-Butoxy-2-hydroxy-propionyl)-2-aza-bicyclo[3.1.0]hexane-3-carboxylic acid 5-chloro-2-tetrazol-1-yl-benzylamide

¹H NMR (500 MHz, CDCl₃): δ 9.01 (s, 1H), 7.59 (d, 1H), 7.44 (dd, 1H), 7.26 (m, 2H), 4.61 (m, 1H), 4.50 (dd, 1H), 4.24 (m, 2H), 3.66 (m, 2H), 3.43 (m, 1H), 2.71 (m, 1H), 2.01-1.89 (m, 2H), 1.20 (s, 9H), 1.08 (m, 1H), 0.61 (m, 1H). HRMS (ESI) calculated for C₂₁H₂₇ClN₆O4 463.1860 (M+H)⁺, found 463.1879.

### Example 25

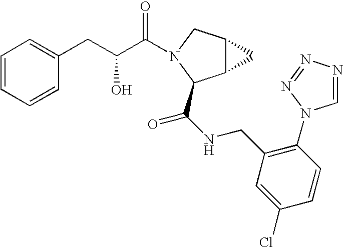

### (1S,2S,5R)-3-((R)-2-Hydroxy-3-phenyl-propionyl)-3-aza-bicyclo[3.1.0]hexane-2-carboxylic acid 5-chloro-2-tetrazol-1-yl-benzylamide

¹H NMR (400 MHz, CDCl₃): δ 8.98 (s, 1H), 7.59 (d, 1H), 7.45 (dd, 1H), 7.34-7.19 (m, 7H), 4.45 (s, 1H), 4.42 (t, 1H), 4.29 (dd, 1H), 4.19 (dd, 1H), 3.63 (dd, 1H), 3.38 (d, 1H), 2.92 (d, 1H), 1.79 (m, 1H), 1.62-1.58 (m, 2H), 0.68 (m, 1H), −0.20 (m, 1H). HRMS (ESI) calculated for C₂₃H₂₃ClN₆O₃ 467.1598 (M+H)⁺, found 467.1562.

### Example 26

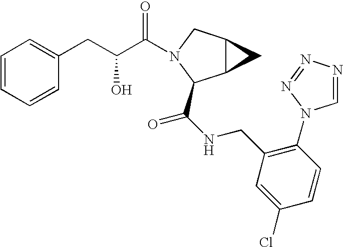

### (1R,2S,5S)-3-((R)-2-Hydroxy-3-phenyl-propionyl)-3-aza-bicyclo[3.1.0]hexane-2-carboxylic acid 5-chloro-2-tetrazol-1-yl-benzylamide

¹H NMR (400 MHz, CDCl₃): δ 9.00 (s, 1H), 7.75 (d, 1H), 7.43 (dd, 1H), 7.32-7.19 (m, 6H), 6.34 (m, 1H), 4.34-4.24 (m, 4H), 3.62 (d, 1H), 3.18 (dd, 1H), 2.96 (dd, 1H), 2.88 (dd, 1H), 1.87 (m, 1H), 1.59 (m, 1H), 0.74 (m, 2H). HRMS (ESI) calculated for C₂₃H₂₃ClN₆O₃ 467.1598 (M+H)⁺, found 467.1589.

### Example 27

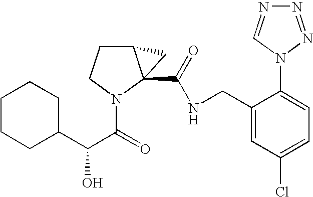

### (1S,5R)-2-((R)-2-Cyclohexyl-2-hydroxy-acetyl)-2-aza-bicyclo[3.1.0]hexane-1-carboxylic acid-5-chloro-2-tetrazol-1-yl-benzylamide

¹H NMR (400 MHz, CDCl₃): 9.01 (s, 1H), 7.77 (s, 1H), 7.41-7.39 (d, 2H ), 4.33-4-28 dd, J=6.2 Hz, 1H), 4.21-4.16 (dd, J=5.4, 1H ), 3.95 (s, 1H), 3.91-3.65 (dt, 2H), 3.15 (s, 1H), 2.34-2.13 (dt, 2H), 1.98-1.95 (t, 1H), 1.74-1.23 (m, 11H), 1.1-0.84 (dd, 2H). HRMS (ESI) calculated for C₂₂H₂₇ClN₆O₃ 459.1911 (M+H)⁺, found 459.1900.

### Example 28

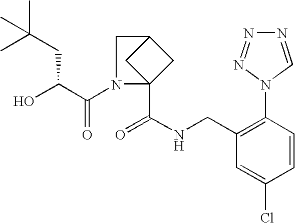

### 2-((R)-2-Hydroxy-4,4-dimethyl-pentanoyl)-2-aza-bicyclo[2.1.1]hexane-1-carboxylic acid 5-chloro-2-tetrazol-1-yl-benzylamide

¹H NMR (500 MHz, CDCl₃): δ 9.09 (s, 1H), 7.83 (d, 1H), 7.45 (dd, 1H), 7.29 (d, 1H), 6.87 (bs, 1H), 4.31 (d, 2H), 4.27 (dd, 1H), 3.54 (m, 2H), 3.20 (bs, 1H), 2.88 (m, 1H), 2.15 (m, 2H), 1.79 (dd, 1H), 1.66 (dd, 1H) 1.53 (dd, 1H), 1.46 (dd, 1H), 1.02 (s, 9H). HRMS (ESI) calculated for C₂₁H₂₇ClN₆O₃ 447.1911 (M+H)⁺, found 447.1937.

### Example 29

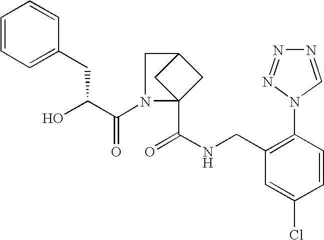

### 2-((R)-2-Hydroxy-3-phenyl-propionyl)-2-aza-bicyclo[2.1.1]hexane-1-carboxylic acid 5-chloro-2-tetrazol-1-yl-benzylamide

¹H NMR (500 MHz, CDCl₃): δ 9.07 (s, 1H), 7.65 (d, 1H), 7.44 (dd, 1H), 7.29-7.18 (m, 6H), 6.43 (bs, 1H), 4.43 (t, 1H), 4.25 (dd, 1H), 4.16 (dd, 1H), 3.65 (bs, 1H), 3.44 (d, 1H), 3.10-2.95 (m, 3H), 2.75 (m, 1H), 2.11 (m, 1H), 2.03 (m, 1H), 1.58-1.48 (m, 2H). HRMS (ESI) calculated for C₂₃H₂₃ClN₆O₃ 467.1589 (M+H)⁺, found 467.1610.

### Example 30

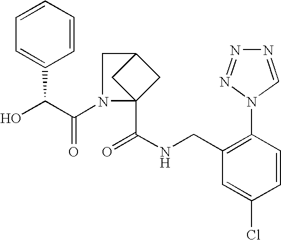

### 2-((R)-2-Hydroxy-2-phenyl-acetyl)-2-aza-bicyclo[2.1.1]hexane-1-carboxylic acid 5-chloro-2-tetrazol-1-yl-benzylamide

¹H NMR (500 MHz, CDCl₃): δ 9.07 (s, 1H), 7.89 (d, 1H), 7.44 (dd, 1H), 7.40-7.30 (m, 6H), 6.78 (bs, 1H), 5.05 (d, 1H), 4.42-4.28 (m, 3H), 3.49 (d, 1H), 3.09 (d, 1H), 2.75 (m, 1H), 2.11 (m, 1H), 2.06 (m, 1H), 1.72 (m, 1H), 1.48 (m, 1H). HRMS (ESI) calculated for C₂₂H₂₁ClN₆O₃ 453.1442 (M+H)⁺, found 453.1460.

### Example 31

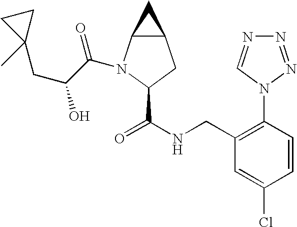

### (1S,3S,5S)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide

¹H NMR (500 MHz, CDCl₃): δ 9.04 (s, 1H), 7.60 (d, 1H), 7.45 (dd, 1H), 7.27 (d, 1H), 4.74 (dd, 1H), 2.59 (dd, 1H), 2.25-2.17 (m, 1H), 1.81 (dd, 1H), 1.48 (dd, 1H), 1.19 (s, 3H), 0.85-0.77 (m, 2H), 0.55-0.50 (m, 1H), 0.43-0.37 (m, 1H), 0.35-0.28 (m, 2H). HRMS (ESI) calculated for C₂₁H₂₅ClN₆O₃ 445.1755 (M+H)⁺, found 445.1766.

### Example 32

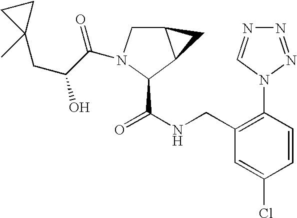

### (1R,2S,5S)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide

¹H NMR (500 MHz, CDCl₃): δ 9.16 (s, 1H), 7.73 (d, 1H), 7.39 (dd, 1H), 7.26 (d, 1H), 6.98 (broad m, 1H), 4.38 (d, 1H), 4.30-4.15 (m, 3H), 3.79 (d, 1H), 3.73-3.67 (m, 1H), 3.40 (broad s, 1H), 1.96-1.90 (m, 1H), 1.80-1.73 (m, 1H), 1.64 (dd, 1H), 1.32 (dd, 1H), 1.11 (s, 3H), 0.84-0.74 (m, 2H), 0.48-0.38 (m, 1H), 0.36-0.31 (m, 1H), 0.28-0.21 (m, 2H). HRMS (ESI) calculated for C₂₁H₂₅ClN₆O₃ 445.1755 (M+H)⁺, found 445.1749.

### Example 33

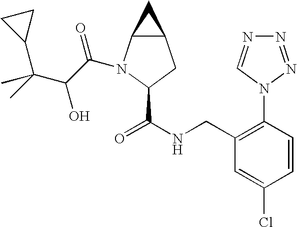

### (1S,3S,5S)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide

¹H NMR (500 MHz, CDCl₃) for the most potent isomer: δ 9.05 (s, 1H), 7.79 (t, 1H), 7.43 (dd, 1H), 7.26 (d, 1H), 4.79 (dd, 1H), 4.39 (s, 1H), 4.20 (d, 2H), 3.74-3.68 (m, 2H), 3.09 (s, 1H), 2.64 (dd, 1H), 0.97 (s, 3H), 0.75 (s, 3H), 0.82-0.68, (m, 2H), 0.36-0.23 (m, 3H). HRMS (ESI) calculated for C₂₂H₂₇ClN₆O₃ 459.1911 (M+H)⁺, found 459.1928.

### Example 34

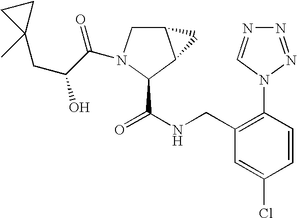

### (1S,2S,5R)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide

¹H NMR (500 MHz, CDCl₃): δ 9.03 (s, 1H), 7.60 (d, 1H), 7.44 (dd, 1H), 7.30 (t, 1H), 4.47 (s, 1H), 4.34-4.15 (m, 3H), 3.70 (dd, 1H), 3.57 (d, 1H), 3.18 (broad s, 1H), 1.85-1.80 (m, 1H), 1.73-1.67 (m, 1H), 1.58 (dd, 1H), 1.30 (dd, 1H), 1.15 (s, 3H), 0.85-0.77 (m, 1H), 0.53-0.47 (m, 1H), 0.40-0.34 (m, 1H), 0.31-0.22 (m, 1H), 0.14-0.09 (m, 1H). HRMS (ESI) calculated for C₂₁H₂₅ClN₆O₃ 445.1755 (M+H)⁺, found 445.1767.

### Example 35

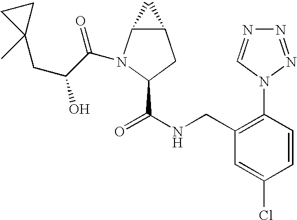

### (1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide

¹H NMR (500 MHz, CDCl₃): δ 9.04 (s, 1H), 7.61 (d, 1H), 7.44 (dd, 1H), 7.31 (t, 1H), 4.67 (m, 1H), 4.41 (dd, 1H), 2.68-2.61 (m, 1H), 2.03-1.97 (m, 1H), 1.97-1.90 (m, 1H), 1.86 (dd, 1H), 1.34 (dd, 1H), 1.17 (s, 3H), 1.13-1.06 (m, 1H), 0.54-0.48 (m, 2H), 0.40-0.35 (m, 1H), 0.31-0.26 (m, 2H). HRMS (ESI) calculated for C₂₁H₂₅ClN₆O₃ 445.1755 (M+H)⁺, found 445.1739.

### Example 36

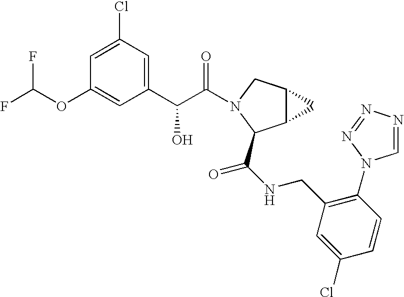

### (1S,2S,5R)-3-[2-((R)-3-Chloro-5-difluoromethoxy-phenyl)-2-hydroxy-acetyl]-3-aza-bicyclo[3.1.0]hexane-2-carboxylic acid 5-chloro-2-tetrazol-1-yl-benzylamide

¹H NMR (400 MHz, CDCl₃): δ 8.98 (s, 1H), 7.64 (d, 1H), 7.48 (dd, 1H), 7.29 (d, 1H), 7.16-7.14 (m, 2H), 7.06 (m, 1H), 6.95 (m, 1H), 6.51 (t, 1H), 5.03 (s, 1H), 4.54 (s, 1H), 4.34 (dd, 1H), 4.25 (dd, 1H), 3.64 (dd, 1H), 3.33 (d, 1H), 1.76 (m, 1H), 1.60 (m, 1H), 0.66 (m, 1H), −0.28 (m, 1H). HRMS (ESI) calculated for C₂₃H₂₀Cl₂F₂N₆O₄ 453.0969 (M+H)⁺, found 453.0950.

### Example 37

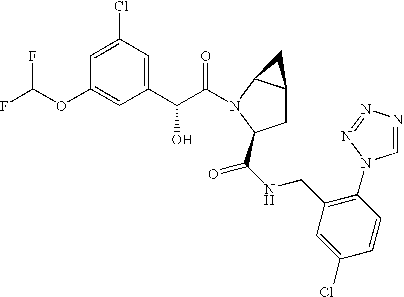

### (1S,3S,5S)-2-[(R)-2-(3-Chloro-5-difluoromethoxy-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃): δ 8.99 (s, 1H), 7.66 (d, 1H), 7.47 (dd, 1H), 7.29-7.06 (m, 5H), 6.52 (t, 1H), 5.29 (s, 1H), 4.75 (dd, 1H), 4.32-4.18 (m, 2H), 3.09 (m, 1H), 2.41 (d, 1H), 2.26 (m, 1H), 1.60 (m, 1H), 0.98 (m, 1H), 0.82 (m, 1H). HRMS (ESI) calculated for C₂₃H₂₀Cl₂F₂N₆O₄ 453.0969 (M+H)⁺, found 453.0967.

### Example 38

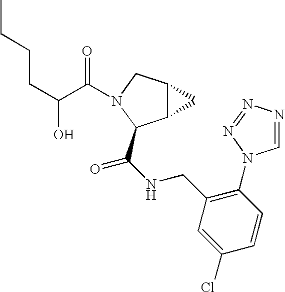

### (1S,2S,5R)-3-(2-Hydroxy-hexanoyl)-3-aza-bicyclo[3.1.0]hexane-2-carboxylic acid 5-chloro-2-tetrazol-1-yl-benzylamide

¹H NMR (400 MHz, CDCl₃) for the most potent isomer: δ 8.98 (s, 1H), 7.59 (d, 1H), 7.45 (dd, 1H), 7.25 (m, 1H), 7.17 (m, 1H), 4.50 (s, 1H), 4.33-4.13 (m, 3H), 3.64 (dd, 1H), 3.58 (d, 1H), 1.86 (m, 1H), 1.70 (m, 1H), 1.52-1.28 (m, 6H), 0.92 (t, 3H), 0.82 (m, 1H), 0.11 (m, 1H). HRMS (ESI) calculated for C₂₀H₂₅ClN₆O₃ 433.1755 (M+H)⁺, found 433.1755.

### Example 39

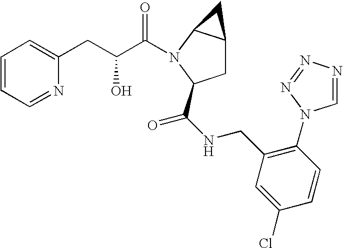

### (1S,3S,5S)-2-((R)-2-Hydroxy-3-pyridin-2-yl-propionyl)-2-aza-bicyclo[3.1.0]hexane-3-carboxylic acid 5-chloro-2-tetrazol-1-yl-benzylamide

¹H NMR (400 MHz, (CD₃)₂CO): δ 9.54 (s, 1H), 8.49 (d, 1H), 7.91 (m, 1H), 7.77 (s, 1H), 7.69 (dt, 1H), 7.58-7.53 (m, 2H), 7.33 (d, 1H), 7.20 (m, 1H), 4.87 (m, 1H), 4.75 (dd, 1H), 4.28 (dd, 1H), 4.19 (dd, 1H), 3.79 (m, 1H), 3.23 (dd, 1H), 3.06 (dd, 1H), 2.39 (m, 1H), 2.11 (dd, 1H), 1.66 (m, 1H), 1.01 (m, 1H), 0.75 (m, 1H). HRMS (ESI) calculated for C₂₂H₂₂ClN₇O₃ 468.1551 (M+H)⁺, found 468.1539.

### Example 40

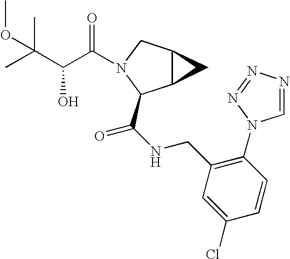

### (1R,2S,5S)-3-(2-Hydroxy-3-methoxy-3-methyl-butyryl)-3-aza-bicyclo[3.1.0]hexane-2-carboxylic acid 5-chloro-2-tetrazol-1-yl-benzylamide

¹H NMR (400 MHz, CDCl₃) for the most potent isomer: δ 8.99 (s, 1H), 7.78 (d, 1H), 7.43 (dd, 1H), 7.25 (m, 1H), 6.10 (m, 1H), 4.41 (d, 1H), 4.31 (dd, 1H), 4.23 (dd, 1H), 4.16 (dd, 1H), 3.97 (d, 1H), 3.67 (d, 1H), 3.19 (s, 3H), 3.15 (d, 1H), 1.89 (m, 1H), 1.71 (m, 1H), 1.28 (s, 3H)m, 1.13 (s, 3H), 0.75 (m, 2H). HRMS (ESI) calculated for C₂₀H₂₅ClN₆O₄ 449.1704 (M+H)⁺, found 449.1700.

### Example 41

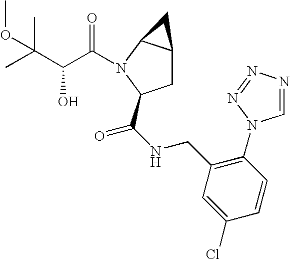

### (1S,3S,5S)-2-(2-Hydroxy-3-methoxy-3-methyl-butyryl)-2-aza-bicyclo[3.1.0]hexane-3-carboxylic acid 5-chloro-2-tetrazol-1-yl-benzylamide

¹H NMR (400 MHz, CDCl₃) for the most potent isomer: δ 9.01 (s, 1H), 7.59 (m, 2H), 7.43 (dd, 1H), 7.25 (m, 1H), 4.80 (dd, 1H), 4.35 (s, 1H), 4.19 (d, 2H), 4.07 (m, 1H), 3.22 (s, 3H), 2.57 (dd, 1H), 2.21 (m, 1H), 1.64 (m, 1H), 1.35 (s, 3H)m, 1.14 (s, 3H), 0.69 (m, 2H). HRMS (ESI) calculated for C₂₀H₂₅ClN₆O₄ 449.1704 (M+H)⁺, found 449.1692.

### Example 42

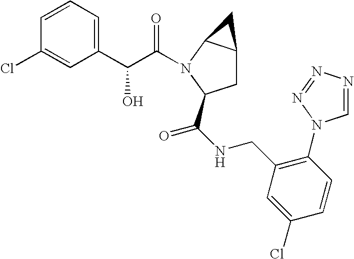

### (1S,3S,5S)-2-[(R)-2-(3-Chloro-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃): δ 9.01 (s, 1H), 7.65 (d, 1H), 7.47 (dd, 1H), 7.39-7.25 (m, 6H), 5.30 (s, 1H), 4.76 (dd, 1H), 4.29 (dd, 1H), 4.20 (dd, 1H), 3.11 (m, 1H), 2.42 (dd, 1H), 2.21 (m, 1H), 1.57 (m, 1H), 0.93 (m, 1H), 0.79 (m, 1H). HRMS (ESI) calculated for C₂₂H₂₀Cl₂N₆O₃ 487.1052 (M+H)⁺, found 487.1055.

### Example 43

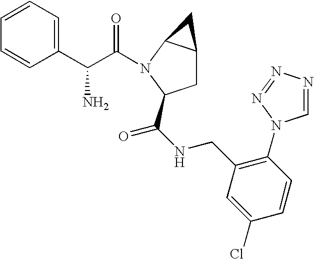

### (1S,3S,5S)-2-((R)-2-Amino-2-phenyl-acetyl)-2-aza-bicyclo[3.1.0]hexane-3-carboxylic acid 5-chloro-2-tetrazol-1-yl-benzylamide

¹H NMR (400 MHz, CD₃CN): δ 9.23 (s, 1H), 7.73 (d, 1H), 7.52 (dd, 1H), 7.45-7.28 (m, 6H), 7.17 (m, 1H), 4.82 (s, 1H), 4.62 (dd, 1H), 4.21 (dd, 1H), 4.10 (dd, 1H), 3.27 (m, 1H), 2.32 (m, 1H), 1.96 (m, 1H), 1.50 (m, 1H), 0.93 (m, 1H), 0.75 (m, 1H). HRMS (ESI) calculated for C₂₂H₂₂ClN₇O₂ 452.1602 (M+H)⁺, found 452.1591.

### Example 44

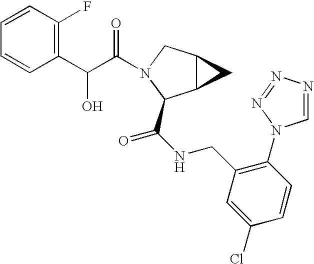

### (1R,2S,5S)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide

¹H NMR (400 MHz, CDCl₃) for the most potent isomer: δ 9.13 (s, 1H), 7.80 (d, 1H), 7.42 (dd, 1H), 7.33-7.25 (m, 3H), 7.16-7.05 (m, 2H), 6.87 (m, 1H), 5.36 (s, 1H), 4.37 (d, 1H), 4.28 (d, 2H), 3.65 (d, 1H), 3.07 (m, 1H), 1.83 (m, 1H), 1.59 (m, 1H), 0.86 (m, 1H), 0.72 (m, 1H). HRMS (ESI) calculated for C₂₂H₂₀ClFN₆O₃ 471.1348 (M+H)⁺, found 471.1349.

### Example 45

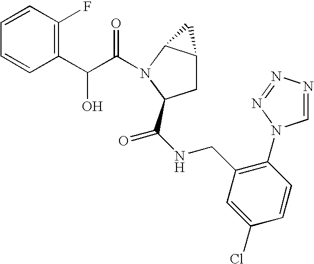

### (1R,3S,5R)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 9.02 (s, 1H), 7.64 (d, 1H), 7.46 (dd, 1H), 7.33-7.04 (m, 6H), 5.70 (d, 1H), 4.40 (m, 1H), 4.27 (d, 2H), 3.24 (m, 1H), 2.54 (m, 1H), 1.88-1.76 (m, 2H), 0.47 (m, 1H), −0.43 (m, 1H). HRMS (ESI) calculated for C₂₂H₂₀ClFN₆O₃ 471.1348 (M+H)⁺, found 471.1343.

### Example 46

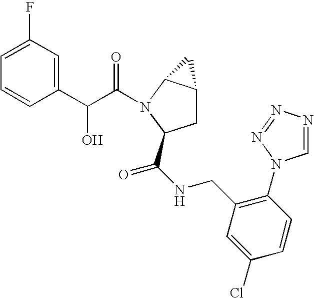

### (1R,3S,5R)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 8.95 (s, 1H), 7.57 (d, 1H), 7.47 (dd, 1H), 7.32-7.26 (m, 2H), 7.11-6.97 (m, 4H), 5.31 (s, 1H), 4.59 (dd, 1H), 4.24 (dd, 1H), 4.14 (dd, 1H), 2.90 (m, 1H), 2.64 (m, 1H), 2.03 (dd, 1H), 1.81 (m, 1H), 1.10 (m, 1H), 0.61 (m, 1H). HRMS (ESI) calculated for C₂₂H₂₀ClFN₆O₃ 471.1348 (M+H)⁺, found 471.1331.

### Example 47

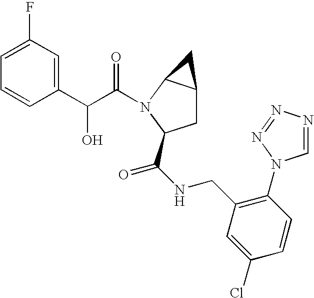

### (1S,3S,5S)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 9.06 (s, 1H), 7.65 (d, 1H), 7.44 (dd, 1H), 7.39-7.01 (m, 6H), 5.31 (s, 1H), 4.75 (dd, 1H), 4.27 (dd, 1H), 4.19 (dd, 1H), 3.12 (m, 1H), 2.34 (dd, 1H), 2.23 (m, 1H), 1.54 (m, 1H), 0.96 (m, 1H), 0.77 (m, 1H). HRMS (ESI) calculated for C₂₂H₂₀ClFN₆O₃ 471.1348 (M+H)⁺, found 471.1345.

### Example 48

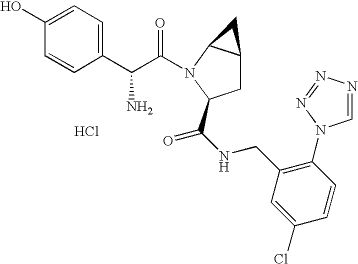

### (1S,3S,5S)-2-[(R)-2-Amino-2-(4-hydroxy-phenyl)-acetyl]-2-aza-bicyclo[3.1.0]hexane-3-carboxylic acid 5-chloro-2-tetrazol-1-yl-benzylamide hydrochloride

¹H NMR (400 MHz, D₂O): δ 9.56 (s, 1H), 7.71 (d, 1H), 7.64 (dd, 1H), 7.53 (d, 1H), 7.43 (d, 2H), 7.01 (d, 2H), 5.47 (s, 1H), 4.80-4.74 (m, 1H), 4.35 (d, 1H), 4.26 (d, 1H), 3.16 (m, 1H), 2.57 (m, 1H), 1.80 (dd, 1H), 1.65 (m, 1H), 0.96-0.88 (m, 2H). HRMS (ESI) calculated for C₂₂H₂₂ClN₇O₃ 468.1551 (M+H)⁺, found 468.1547.

### Example 49

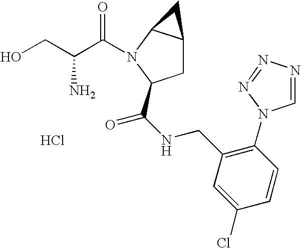

### (1S,3S,5S)-2-((R)-2-Amino-3-hydroxy-propionyl)-2-aza-bicyclo[3.1.0]hexane-3-carboxylic acid 5-chloro-2-tetrazol-1-yl-benzylamide hydrochloride

¹H NMR (400 MHz, D₂O): δ 9.54 (s, 1H), 7.68 (d, 1H), 7.63 (dd, 1H), 7.52 (d, 1H), 4.73 (dd, 1H), 4.62 (dd, 1H), 4.34 (d, 1H), 4.23 (d, 1H), 4.09 (dd, 1H), 4.00 (dd, 1H), 3.61 (m, 1H), 2.68 (m, 1H), 1.91-1.83 (m, 2H), 0.99-0.89 (m, 2H). HRMS (ESI) calculated for C₁₇H₂₀ClN₇O₃ 406.1394 (M+H)⁺, found 406.1418.

### Example 50

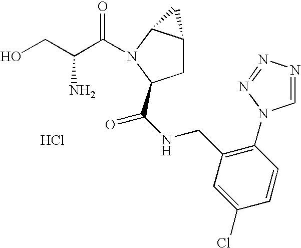

### (1R,3S,5R)-2-((R)-2-Amino-3-hydroxy-propionyl)-2-aza-bicyclo[3.1.0]hexane-3-carboxylic acid 5-chloro-2-tetrazol-1-yl-benzylamide hydrochloride

¹H NMR (400 MHz, D₂O): δ 9.54 (s, 1H), 7.69 (d, 1H), 7.63 (dd, 1H), 7.52 (d, 1H), 4.70 (m, 1H), 4.30 (d, 2H), 4.25 (m, 1H), 4.18 (dd, 1H), 4.11 (dd, 1H), 3.63 (m, 1H), 2.39 (m, 1H), 2.08 (m, 1H), 1.99 (m, 1H), 1.11 (m, 1H), 0.72 (m, 1H). HRMS (ESI) calculated for C₁₇H₂₀ClN₇O₃ 406.1394 (M+H)⁺, found 406.1428.

### Example 51

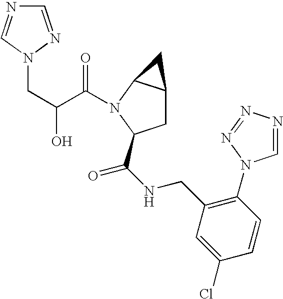

### (1S,3S,5S)-2-(2-Hydroxy-3-1,2,4-triazol-1-yl-propionyl)-2-aza-bicyclo3.1.0]hexane-3-carboxylic acid 5-chloro-2-tetrazol-1-yl-benzylamide

¹H NMR (400 MHz, CDCl₃) for the most potent isomer: δ 9.03 (s, 1H), 8.21 (s, 1H), 7.89 (s, 1H), 7.60 (d, 1H), 7.44-7.40 (m, 2H), 7.26 (d, 1H), 4.86 (m, 1H), 4.70 (dd, 1H), 4.52 (dd, 1H), 4.44 (dd, 1H), 4.17 (d, 2H), 3.46 (m, 1H), 2.39 (dd, 1H), 2.28 (m, 1H), 1.70 (m, 1H), 1.55 (m, 1H), 0.86-0.77 (m, 2H). HRMS (ESI) calculated for C₁₉H₂₀ClN₉O₃ 458.1456 (M+H)⁺, found 458.1429.

### Example 52

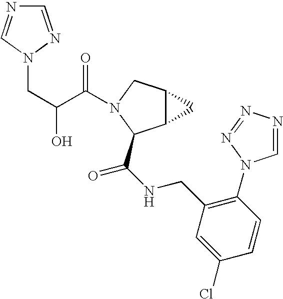

### (1S,2S,5R)-3-(2-Hydroxy-3-1,2,4-triazol-1-yl-propionyl)-3-aza-bicyclo[3.1.0]hexane-2-carboxylic acid 5-chloro-2-tetrazol-1-yl-benzylamide

¹H NMR (400 MHz, CDCl₃) for the most potent isomer: δ 8.95 (s, 1H), 8.17 (s, 1H), 7.91 (s, 1H), 7.60 (d, 1H), 7.47 (dd, 1H), 7.28-7.26 (m, 1H), 6.96 (m, 1H), 4.58 (m, 1H), 4.47-4.17 (m, 5H), 3.78 (dd, 1H), 3.71 (d, 1H), 1.83 (m, 1H), 1.71 (m, 1H), 0.83 (m, 1H), 0.25 (m, 1H). HRMS (ESI) calculated for C₁₉H₂₀ClN₉O₃ 458.1456 (M+H)⁺, found 458.1440.

### Example 53

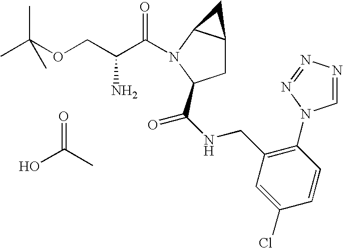

### (1S,3S,5S)-2-((R)-2-Amino-3-tert-butoxy-propionyl)-2-aza-bicyclo[3.1.0]hexane-3-carboxylic acid 5-chloro-2-tetrazol-1-yl-benzylamide acetate

¹H NMR (400 MHz, CDCl₃): δ 9.11 (s, 1H), 7.76 (m, 1H), 7.60 (d, 1H), 7.42 (dd, 1H), 7.24 (m, 1H), 4.82 (dd, 1H), 4.23-4.06 (m, 3H), 3.74-3.69 (m, 2H), 3.53-3.42 (m, 2H), 2.93 (m, 1H), 2.51 (dd, 1H), 2.27 (m, 1H), 1.66 (m, 1H), 1.17 (s, 9H), 0.77 (m, 1H), 0.66 (m, 1H). HRMS (ESI) calculated for C₂₁H₂₈ClN₇O₃ 462.2020 (M+H)+, found 462.2022.

### Example 54

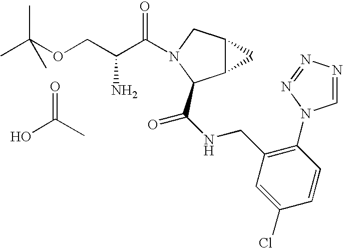

### (1S,2S,5R)-3-((R)-2-Amino-3-tert-butoxy-propionyl)-3-aza-bicyclo[3.1.0]hexane-2-carboxylic acid 5-chloro-2-tetrazol-1-yl-benzylamide acetate

¹H NMR (400 MHz, CDCl₃): δ 9.08 (s, 1H), 7.69 (m, 1H), 7.58 (d, 1H), 7.42 (dd, 1H), 7.25 (m, 1H), 4.58 (s, 1H), 4.28-4.11 (m, 2H), 3.86 (d, 1H), 3.80-3.72 (m, 2H), 3.42-3.29 (m, 2H), 1.86 (m, 1H), 1.63 (m, 1H), 1.18 (s, 9H), 0.81 (m, 1H), 0.21 (m, 1H). HRMS (ESI) calculated for C₂₁H₂₈ClN₇O₃ 462.2020 (M+H)⁺, found 462.2022.

### Example 55

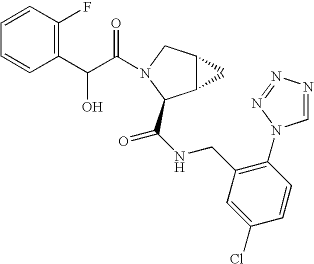

### (1S,2S,5R)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide

¹H NMR (400 MHz, CDCl₃) for the major rotamer of the most potent isomer: δ 9.00 (s, 1H), 7.63 (d, 1H), 7.48 (dd, 1H), 7.37-7.09 (m, 6H), 5.39 (s, 1H), 4.53 (s, 1H), 4.32 (dd, 1H), 4.25 (dd, 1H), 3.60 (dd, 1H), 3.32 (d, 1H), 1.76 (m, 1H), 1.55 (m, 1H), 0.58 (m, 1H), −0.37 (m, 1H). HRMS (ESI) calculated for C₂₂H₂₀ClFN₆O₃ 471.1348 (M+H)⁺, found 471.1333.

### Example 56

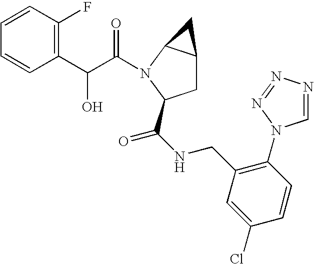

### (1S,3S,5S)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 9.04 (s, 1H), 7.65 (d, 1H), 7.45 (dd, 1H), 7.41-7.31 (m, 3H), 7.27 (d, 1H), 7.19-7.08 (m, 2H), 5.67 (d, 1H), 4.73 (dd, 1H), 4.33 (d, 1H), 4.27 (dd, 1H), 4.20 (dd, 1H), 3.13 (m, 1H), 2.40 (dd, 1H), 2.19 (m, 1H), 1.54 (m, 1H), 0.91 (m, 1H), 0.78 (m, 1H). HRMS (ESI) calculated for C₂₂H₂₀ClFN₆O₃ 471.1348 (M+H)⁺, found 471.1342.

### Example 57

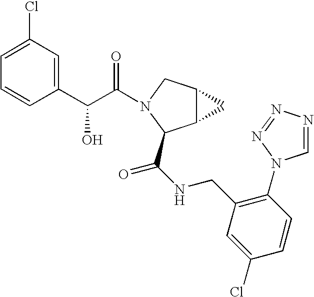

### (1S,2S,5R)-3-[(R)-2-(3-Chloro-phenyl)-2-hydroxy-acetyl]-3-aza-bicyclo[3.1.0]hexane-2-carboxylic acid 5-chloro-2-tetrazol-1-yl-benzylamide

¹H NMR (400 MHz, CDCl₃) for the major rotamer: δ 9.07 (s, 1H), 7.62 (d, 1H), 7.43 (dd, 1H), 7.37-7.13 (m, 6H), 5.00 (d, 1H), 4.52 (s, 1H), 4.41 (d, 1H), 4.29 (dd, 1H), 4.23 (dd, 1H), 3.62 (dd, 1H), 3.31 (d, 1H), 1.69 (m, 1H), 1.52 (m, 1H), 0.57 (m, 1H), −0.35 (m, 1H). HRMS (ESI) calculated for C₂₂H₂₀Cl₂N₆O₃ 487.1052 (M+H)⁺, found 487.1030.

### Example 58

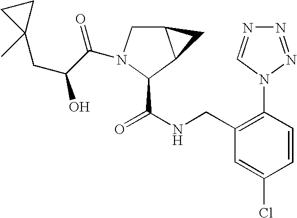

### (1R,2S,5S)-3-[(S)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide

¹H NMR (500 MHz, CDCl₃): δ 9.04 (s, 1H), 7.74 (d, 1H), 7.44 (dd, 1H), 7.27 (d, 1H), 6.62 (t, 1H), 4.44 (d, 1H), 4.33 (d, 2H), 3.78 (dd, 1H), 3.61 (d, 1H), 3.25 (broad s, 1H), 1.94-1.87 (m, 1H), 1.81-1.74 (m, 1H), 1.69 (dd, 1H), 1.31 (dd, 1H), 1.16 (s, 3H), 0.82-0.73 (m, 3H), 0.56-0.50 (m, 1H), 0.38-0.25 (m, 2H). HRMS (ESI) calculated for C₂₁H₂₅ClN₆O₃ 445.1755 (M+H)⁺, found 445.1766.

### Example 59

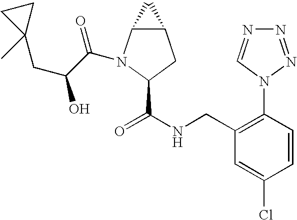

### (1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide

¹H NMR (500 MHz, CDCl₃): δ 9.05 (s, 1H), 7.55 (d, 1H), 7.46 (dd, 1H), 7.35 (t, 1H), 4.68 (m, 1H), 4.60 (dd, 1H), 2.79-2.72 (m, 1H), 2.05-1.98 (m, 1H), 1.97-1.90 (m, 1H), 1.81 (dd, 1H), 1.45 (dd, 1H), 1.19 (s, 3H), 1.16-1.11 (m, 1H), 0.64-0.60 (m, 1H), 0.58-0.53 (m, 1H), 0.40-0.35 (m, 1H), 0.34-0.27 (m, 2H). HRMS (ESI) calculated for C₂₁H₂₅ClN₆O₃ 445.1755 (M+H)⁺, found 445.1758.

### Example 60

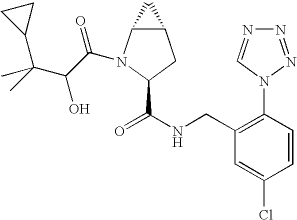

### (1R,3S,5R)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide

¹H NMR (500 MHz, CDCl₃) for the most potent isomer: δ 9.10 (s, 1H), 7.62 (d, 1H), 7.41 (dd, 1H), 7.39 (broad s, 1H), 7.25 (d, 1H), 4.42 (dd, 1H), 4.32 (s, 1H), 4.26 (m, 2H), 3.54-3.50 (m, 1H), 3.02 (broad s, 1H), 2.58-2.51 (m, 1H), 2.03-1.97 (m, 1H), 1.94-1.87 (m, 1H), 1.10-1.04 (m, 1H), 0.94 (s, 3H), 0.93-0.89, (m, 1H), 0.73 (s, 3H), 0.57-0.53, (m, 1H), 0.35-0.20 (m, 4H). HRMS (ESI) calculated for C₂₂H₂₇ClN₆O₃ 459.1911 (M+H)⁺, found 459.1905.

### Example 61

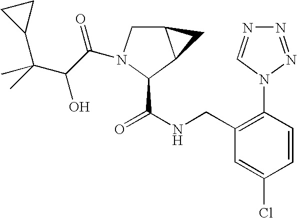

### (1R,2S,5S)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide

¹H NMR (500 MHz, CDCl₃) for the most potent isomer: δ 9.06 (s, 1H), 7.79 (d, 1H), 7.27 (d, 1H), 6.34 (t, 1H), 4.42 (d, 1H), 4.37-4.22 (m, 2H), 3.98-3.94 (m, 2H), 3.81 (d, 1H), 2.97 (s, broad, 1H), 1.97-1.90 (m, 1H), 1.77-1.71 (m, 1H), 0.94 (s, 3H), 0.71 (s, 3H), 0.81-0.75, (m, 3H), 0.36-0.20 (m, 4H). HRMS (ESI) calculated for C₂₂H₂₇ClN₆O₃ 459.1911 (M+H)⁺, found 459.1905.

### Example 62

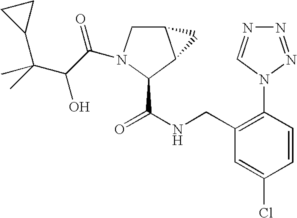

### (1S,2S,5R)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide

¹H NMR (500 MHz, CDCl₃) for the most potent isomer: δ 9.08 (s, 1H), 7.63 (d, 1H), 7.40 (t, 1H), 7.27 (d, 1H), 4.55 (s, 1H), 4.32-4.18 (m, 2H), 4.03 (s, 1H), 3.94 (d, 1H), 3.77 (dd, 1H), 7.56 (dd, 1H), 7.47 (d, 1H), 7.24-7.20 (m, 1H), 7.13 (dd, 1H), 7.10 (t, 1H), 6.93 (td, 1H), 6.87 (d, 1H), 5.12 (s, 1H), 4.73 (dd, 1H), 4.43-4.39 (m, 1H), 4.35 (dd, 1H), 4.23-4.17 (m, 1H), 4.09 (m, 1H), 2.85-2.80 (m, 1H), 2.53-2.46 (m, 1H), 2.45-2.37 (m, 2H), 1.98-1.93 (m, 1H), 1.81 (dd, 1H), 1.54-1.48 (m, 1H), 0.69-0.64 (m, 1H), −0.065-(−0.12) (m, 1H). HRMS (ESI) calculated for C₂₄H₂₃ClN₆O₄ 495.1548 (M+H)⁺, found 495.1533.

### Example 64

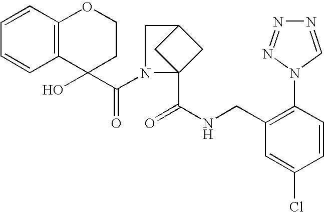

### (rac)-2-(4-Hydroxy-1-benzopyran-4-carbonyl)-2-aza-bicyclo[2.1.1]hexane-1-carboxylic acid 5-chloro-2-tetrazol-1-yl-benzylamide

¹H NMR (500 MHz, CDCl₃): δ 9.06 (s, 1H), 7.92 (d, 1H), 7.48 (d, 1H), 7.31 (d, 1H), 7.22 (m, 1H), 7.08 (m, 1H), 6.92 (m, 2H), 6.37 (m, 1H), 5.18 (bs, 1H), 4.42-4.36 (m, 3H), 4.22 (m, 1H), 3.24 (d, 1H), 2.60 (m, 1H), 2.40 (m, 2H), 2.08-2.00 (m, 3H), 1.71 (dd, 1H), 1.45 (dd, 1H). HRMS (ESI) calculated for C₂₄H₂₃ClN₆O₄ 495.1548 (M+H)⁺, found 495.1544.

### Example 65

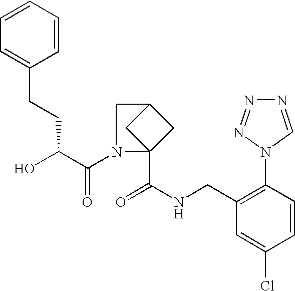

### 2-((R)-2-Hydroxy-4-phenyl-butyryl)-2-aza-bicyclo[2.1.1]hexane-1-carboxylic acid 5-chloro-2-tetrazol-1-yl-benzylamide

¹H NMR (500 MHz, CDCl₃): δ 9.04 (s, 1H), 7.83 (d, 1H), 7.44 (dd, 1H), 7.31-7.18 (m, 6H), 6.70 (bs, 1H), 4.33 (dd, 1H), 4.27 (dd, 1H), 4.13 (m, 1H), 3.49 (d, 1H), 3.38 (m, 2H), 2.90-2.70 (m, 3H), 2.15-2.08 (m, 2H), 2.00-1.85 (m, 2H), 1.72 (m, 1H), 1.59 (m, 1H). HRMS (ESI) calculated for C₂₄H₂₅ClN₆O₃ 481.1755 (M+H)⁺, found 481.1739.

### Example 66

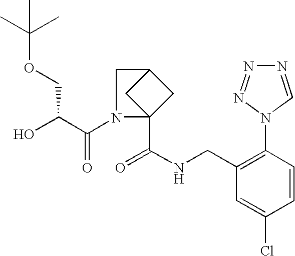

### 2-((R)-3-tert-Butoxy-2-hydroxy-propionyl)-2-aza-bicyclo[2.1.1]hexane-1-carboxylic acid 5-chloro-2-tetrazol-1-yl-benzylamide

¹H NMR (500 MHz, CDCl₃): δ 9.10 (s, 1H), 7.81 (d, 1H), 7.45 (dd, 1H), 7.29 (d, 1H), 6.87 (m, 1H), 4.31 (m, 3H), 3.86 (m, 1H), 3.65-3.57 (m, 2H), 3.51 (dd, 1H), 3.38 (bs, 1H), 2.86 (m, 1H), 2.15 (m, 2H), 1.74 (dd, 1H), 1.67 (dd, 1H), 1.17 (s, 9H). HRMS (ESI) calculated for C₂₁H₂₇ClN₆O₄ 463.1860 (M+H)⁺, found 463.1842.

### Example 67

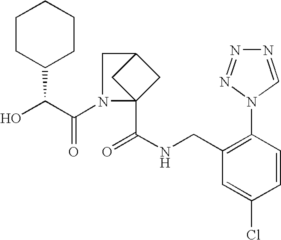

### 2-((R)-2-Cyclohexyl-2-hydroxy-acetyl)-2-aza-bicyclo[2.1.1]hexane-1-carboxylic acid 5-chloro-2-tetrazol-1-yl-benzylamide

¹H NMR (500 MHz, CDCl₃): δ 9.04 (s, 1H), 7.81 (d, 1H), 7.42 (dd, 1H), 7.25 (d, 1H), 6.73 (bs, 1H), 4.37 (dd, 1H), 4.19 (dd, 1H), 3.97 (m, 1H), 3.59 (d, 1H), 3.53 (d, 1H), 3.25 (bs, 1H), 2.85 (m, 1H), 2.11 (m, 2H), 1.80-1.05 (m, 13H). HRMS (ESI) calculated for C₂₂H₂₇ClN₆O₃ 459.1911 (M+H)⁺, found 459.1906.

### Example 68

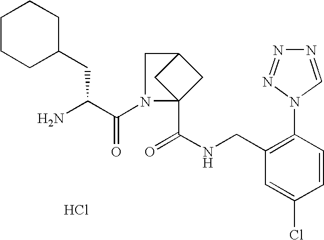

### 2-((R)-2-Amino-3-cyclohexyl-propionyl)-2-aza-bicyclo[2.1.1]hexane-1-carboxylic acid 5-chloro-2-tetrazol-1-yl-benzylamide hydrochloride

¹H NMR (500 MHz, CD₃OD): δ 9.56 (s, 1H), 7.83 (d, 1H), 7.56 (dd, 1H), 7.50 (d, 1H), 4.30 (q, 16.0 Hz, 2H), 4.13 (m, 1H), 3.68 (d, 1H), 3.61 (d, 1H), 2.90 (m, 1H), 2.17 (m, 1H), 2.12 (m, 1H), 1.90 (m, 1H), 1.82-1.65 (m, 8H), 1.50-0-95 (m, 6H). HRMS (ESI) calculated for C₂₃H₃₀ClN₇O₂×HCl 472.2228 (M+H)⁺, found 472.2214.

### Example 69

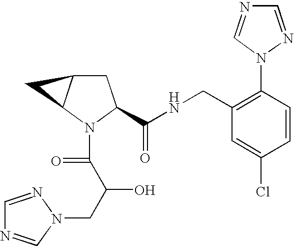

### (1S,3S,5S)-2-(2-Hydroxy-3-1,2,4-triazol-1-yl-propionyl)-2-aza-bicyclo[3.1.0]hexane-3-carboxylic acid 5-chloro-2-1,2,4-triazol-1-yl-benzylamide

¹H NMR (500 MHz, CD₃OD) for the most potent isomer: δ 8.82 (s, 1H), 8.52 (s, 1H), 8.22 (s, 1H), 8.01 (s, 1H), 7.65 (d, 1H) 7.48 (dd, 1H), 7.43 (d, 1H), 4.72 (dd, 1H), 4.60 (dd, 1H), 4.47 (m, 1H), 4.28 (dd, 2H), 3.71 (m, 1H), 2.54 (m, 1H), 1.94 (dd, 1H),), 1.74 (m, 1H), 1.02 (m, 1H), 0.83 (m, 1H). HRMS (ESI) calculated for C₂₀H₂₁ClN₈O₃ 457.1503 (M+H)⁺, found 457.1502.

### Example 70

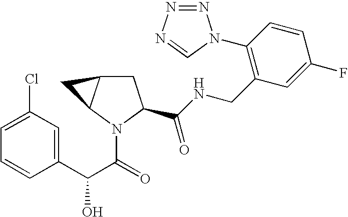

### (1S,3S,5S)-2-[(R)-2-(3-Chloro-phenyl)-2-hydroxy-acetyl]-2-aza-bicyclo[3.1.0]hexane-3-carboxylic acid 5-fluoro-2-tetrazol-1-yl-benzylamide

¹H NMR (500 MHz, CDCl₃): δ 9.06 (s, 1H), 7.41-7.27 (s, 7H), 7.20-7.16 (m, 1H), 4.77 (dd, 1H), 4.28-4.18 (m, 2H), 3.14-3.11 (m, 1H), 2.36 (dd, J=2.7 Hz, 1H), 2.29-2.23 (m, 1H), 1.60-1.54 (m, 1H), 0.99-0.95 (m, 1H), 0.82-0.78 (m, 1H). HRMS (ESI) calculated for C₂₀H₂₅ClN₆O₃ 433.1755 (M+H)⁺, found 433.1757.

### Example 71

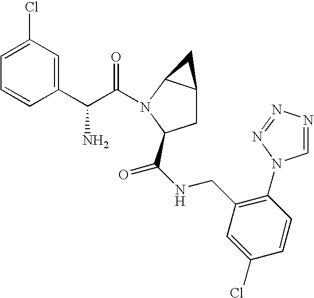

### (1S,3S,5S)-2-[(R)-2-Amino-2-(3-chloro-phenyl)-acetyl]-2-aza-bicyclo[3.1.0]hexane-3-carboxylic acid 5-chloro-2-tetrazol-1-yl-benzylamide

¹H NMR (400 MHz, CDCl₃): δ 9.10 (s, 1H), 7.63 (d, 1H), 7.52 (m, 1H), 7.44 (dd, 1H), 7.38 (s, 1H), 7.31-7.25 (m, 4H), 4.84 (m, 1H), 4.77 (dd, 1H), 4.26 (dd, 1H), 4.17 (dd, 1H), 3.19 (m, 1H), 2.41 (dd, 1H), 2.18 (m, 1H), 1.54 (m, 1H), 0.87-0.75 (m, 2H). HRMS (ESI) calculated for C₂₂H₂₁Cl₂N₇O₂ 486.1212 (M+H)⁺, found 486.1215.

### Example 72

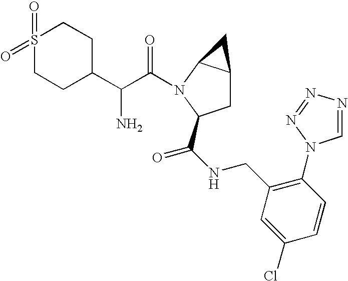

### (1S,3S,5S)-2-[2-Amino-2-(1,1-dioxo-hexahydro-1λ⁶-thiopyran-4-yl)-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 8.98 (s, 1H), 7.62 (d, 1H), 7.46 (dd, 1H), 7.28-7.26 (m, 2H), 4.75 (dd, 1H), 4.24 (dd, 1H), 4.15 (dd, 1H), 3.59 (d, 1H), 3.37 (m, 1H), 3.15-2.95 (m, 4H), 2.56 (m, 1H), 2.47 (dd, 1H), 2.29 (m, 1H) 2.12-1.91 (m, 3H), 1.84-1.71 (m, 2H), 0.91 (m, 1H), 0.81 (m, 1H). HRMS (ESI) calculated for C₂₁H₂₆ClN₇O₄S 508.1534 (M+H)⁺, found 508.1535.

### Example 73

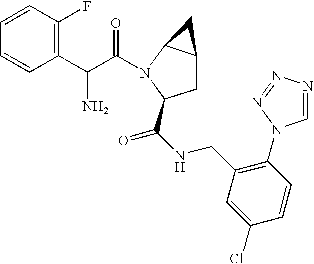

### (1S,3S,5S)-2-[2-Amino-2-(2-fluoro-phenyl)-acetyl]-2-aza-bicyclo[3.1.0]hexane-3-carboxylic acid 5-chloro-2-tetrazol-1-yl-benzylamide

¹H NMR (400 MHz, CDCl₃) for the most potent isomer: δ 9.10 (s, 1H), 7.62 (d, 1H), 7.55 (m, 1H), 7.44 (dd, 1H), 7.42-7.07 (m, 5H), 5.24 (s, 1H), 4.76 (dd, 1H), 4.25 (dd, 1H), 4.18 (dd, 1H), 3.28 (m, 1H), 2.49 (dd, 1H), 2.12 (m, 1H) 1.54 (m, 1H), 0.81-0.75 (m, 2H). HRMS (ESI) calculated for C₂₂H₂₁ClFN₇O₂ 470.1507 (M+H)⁺, found 470.1516.

### Example 74

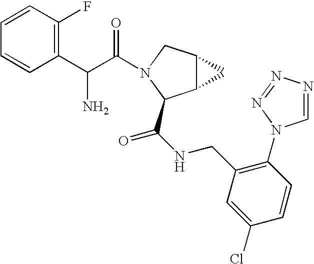

### (1S,2S,5R)-3-[2-Amino-2-(2-fluoro-phenyl)-acetyl]-3-aza-bicyclo[3.1.0]hexane-2-carboxylic acid 5-chloro-2-tetrazol-1-yl-benzylamide

¹H NMR (400 MHz, CDCl₃) for the major rotamer of the most potent isomer: δ 9.07 (s, 1H), 7.61 (d, 1H), 7.47 (dd, 1H), 7.35-7.07 (m, 6H), 4.91 (s, 1H), 4.55 (s, 1H), 4.31 (dd, 1H), 4.22 (dd, 1H), 3.62 (dd, 1H), 3.43 (d, 1H), 1.80 (m, 1H) 1.54 (m, 1H), 0.58 (m, 1H), −0.35 (m, 1H). HRMS (ESI) calculated for C₂₂H₂₁ClFN₇O₂ 470.1507 (M+H)⁺, found 470.1510.

### Example 75

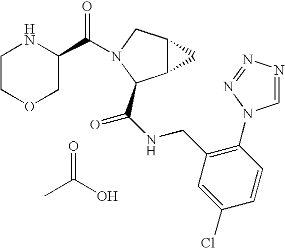

### (1S,2S,5R)-3-((R)-Morpholine-3-carbonyl)-3-aza-bicyclo[3.1.0]hexane-2-carboxylic acid 5-chloro-2-tetrazol-1-yl-benzylamide acetate

¹H NMR (600 MHz, CDCl₃): δ 9.06 (s, 1H), 7.58 (d, 1H), 7.48-7.42 (m, 2H), 7.25 (d, 1H), 4.51 (s, 1H), 4.24 (dd, 1H), 4.16 (dd, 1H), 3.87-3.74 (m, 4H), 3.64 (d, 1H), 3.47 (m, 1H), 3.36 (t, 1H), 2.97 (d, 2H), 1.88 (m, 1H), 1.66 (m, 1H), 0.84 (m, 1H), 0.07 (m, 1H). HRMS (ESI) calculated for C₁₉H₂₂ClN₇O₃ 432.1551 (M+H)⁺, found 432.1551.

### Example 76

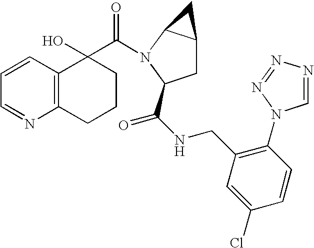

### (1S,3S,5S)-2-(5-Hydroxy-5,6,7,8-tetrahydro-quinoline-5-carbonyl)-2-aza-bicyclo[3.1.0]hexane-3-carboxylic acid 5-chloro-2-tetrazol-1-yl-benzylamide

¹H NMR (600 MHz, CDCl₃) for the less potent isomer: δ 9.00 (s, 1H), 8.48 (d, 1H), 7.77 (d, 1H), 7.49 (dd, 1H), 7.39 (m, 1H), 7.30 (d, 1H), 7.20 (m, 1H), 6.97 (m, 1H), 4.76 (dd, 1H), 4.39 (dd, 1H), 4.18 (dd, 1H), 3.17 (d, 1H), 2.87 (m, 1H), 2.65 (m, 1H), 2.30 (m, 1H), 2.21 (m, 1H), 2.16 (dd, 1H), 2.10-2.05 (m, 3H), 1.55 (m, 1H), 0.63 (m, 1H), −0.02 (m, 1H). HRMS (ESI) calculated for C₂₄H₂₄ClN₇O₃ 494.1707 (M+H)⁺, found 494.1723.

### Example 77

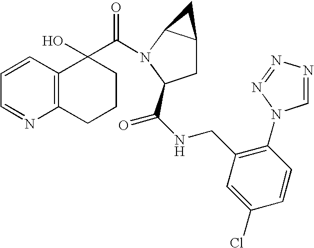

### (1S,3S,5S)-2-(5-Hydroxy-5,6,7,8-tetrahydro-quinoline-5-carbonyl)-2-aza-bicyclo[3.1.0]hexane-3-carboxylic acid 5-chloro-2-tetrazol-1-yl-benzylamide

¹H NMR (600 MHz, CDCl₃) for the most potent isomer: δ 8.99 (s, 1H), 8.51 (dd, 1H), 7.63 (d, 1H), 7.47 (dd, 1H), 7.41 (dd, 1H), 7.29 (d, 1H), 7.17 (dd, 1H), 7.07 (m, 1H), 4.86 (dd, 1H), 4.30 (dd, 1H), 4.15 (dd, 1H), 3.13 (d, 1H), 2.89 (m, 1H), 2.52 (dd, 1H), 2.46 (m, 1H), 2.29 (dd, 1H), 2.21 (m, 2H), 2.09 (m, 1H), 1.98 (d, 1H), 1.46 (m, 1H), 0.96 (m, 1H), 0.68 (m, 1H). HRMS (ESI) calculated for C₂₄H₂₄ClN₇O₃ 494.1707 (M+H)⁺, found 494.1721.

Biological Tests

The following test procedures may be employed

Test A

Determination of Thrombin Inhibition with a Chromogenic, Robotic Assay

The thrombin inhibitor potency is measured with a chromogenic substrate method, in a Plato 3300 robotic microplate processor (Rosys AG, CH-8634 Hombrechtikon, Switzerland), using 96-well, half volume microtitre plates (Costar, Cambridge, Mass., USA; Cat No 3690). Stock solutions of test substance in DMSO (72 μL), 0.1-1 mmol/L, are diluted serially 1:3 (24+48 μL) with DMSO to obtain ten different concentrations, which are analysed as samples in the assay. 2 μL of test sample is diluted with 124 μL assay buffer, 12 μL of chromogenic substrate solution (S-2366, Chromogenix, Mölndal, Sweden) in assay buffer and finally 12 μL of α-thrombin solution (Human α-thrombin, Sigma Chemical Co. or Hematologic Technologies) in assay buffer, are added, and the samples mixed. The final assay concentrations are: test substance 0.00068-133 μmol/L, S-2366 0.30 mmol/L, α-thrombin 0.020 NIHU/mL. The linear absorbance increment during 40 minutes incubation at 37° C. is used for calculation of percentage inhibition for the test samples, as compared to blanks without inhibitor. The IC₅₀ value, corresponding to the inhibitor concentration which causes 50% inhibition of the thrombin activity, is calculated from a log concentration vs. % inhibition curve.

Test B

Determination of Activated Partial Thromboplastin Time (APTT)

APTT is determined in pooled normal human citrated plasma with the reagent PTT Automated 5 manufactured by Stago. The inhibitors are added to the plasma (10 μL inhibitor solution to 90 μL plasma) and incubated with the APTT reagent for 3 minutes followed by the addition of 100 μL of calcium chloride solution (0.025 M) and APTT is determined by use of the coagulation analyser KC10 (Amelung) according to the instructions of the reagent producer.

The clotting time is expressed as absolute values (seconds) as well as the ratio of APTT without inhibitor (APTT₀) to APTT with inhibitor (APTTᵢ). The latter ratios (range 1-0) are plotted against the concentration of inhibitor (log transformed) and fitted to sigmoidal dose-response curves according to the equation

y=a/[1+(x/IC₅₀)^{s}]

where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).

IC₅₀APTT is defined as the concentration of inhibitor in human plasma that doubled the Activated Partial Thromboplastin Time.

Results

Compounds of the Examples were tested in Test A as described above and were found to exhibit IC₅₀ values of less than 1 μM. The following table shows the IC₅₀ values for a representative selection of compounds:

| Example | Test A |
|---|---|
| No. | IC₅₀ (nM) |
| 1 | 5 |
| 2 | 9 |
| 3 | 3 |
| 4 | 3 |
| 5 | 3 |
| 6 | 10 |
| 7 | 14 |
| 8 | 11 |
| 9 | 3 |
| 10 | 6 |
| 11 | 6 |
| 12 | 4 |
| 13 | 2 |
| 14 | 5 |
| 15 | 33 |
| 16 | 52 |
| 17 | 56 |
| 18 | 1 |
| 19 | 4 |
| 20 | 2 |
| 21 | 2 |
| 22 | 4 |
| 23 | 5 |
| 24 | 4 |
| 25 | 18 |
| 26 | 8 |
| 27 | 7 |
| 28 | 6 |
| 29 | 290 |
| 30 | 47 |
| 31 | 9 |
| 32 | 10 |
| 33 | 10 |
| 34 | 8 |
| 35 | 10 |
| 36 | 9 |
| 37 | 4 |
| 38 | 8 |
| 39 | 16 |
| 40 | 11 |
| 41 | 16 |
| 42 | 2 |
| 43 | 5 |
| 44 | 4 |
| 45 | 7 |
| 46 | 410 |
| 47 | 6 |
| 48 | 22 |
| 49 | 450 |
| 50 | 2500 |
| 51 | 420 |
| 52 | 570 |
| 53 | 5 |
| 54 | 6 |
| 55 | 20 |
| 56 | 6 |
| 57 | 3 |
| 58 | 340 |
| 59 | 240 |
| 60 | 14 |
| 61 | 15 |
| 62 | 2 |
| 63 | 41 |
| 64 | 40 |
| 65 | 170 |
| 66 | 67 |
| 67 | 85 |
| 68 | 4 |
| 69 | 780 |
| 70 | 36 |
| 71 | 5 |
| 72 | 240 |
| 73 | 8 |
| 74 | 29 |
| 75 | 650 |
| 76 | 11 |
| 77 | 9 |

1. A compound of formula (I) 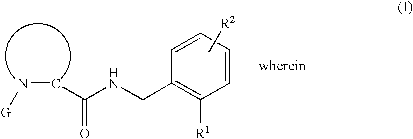 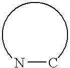
forms an aza-bicyclo[3.1.0]hexane, or 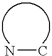
forms an aza-bicyclo[2.1.1]hexane;
R¹ is a 5-membered heteroaryl ring containing 2, 3 or 4 heteroatoms, selected from N, O and S, wherein at least 2 heteroatoms are N, and 0 or 1 heteroatoms are O or S, wherein said 5-membered heteroaryl ring is substituted, at any carbon ring atom, by 0, 1 or 2 substituents independently selected from C₁₋₆ alkyl and a 6-membered heteroaryl ring containing 1 or 2 nitrogen atoms, wherein said 6-membered heteroaryl ring is substituted, at any carbon ring atom, by 0, 1, 2 or 3 substituents independently selected from C₁₋₆ alkyl;
R² is H, halogen, cyano, C₁₋₆ alkyl or C₁₋₆ alkoxy, wherein said C₁₋₆ alkyl or C₁₋₆ alkoxy is substituted by 0, 1, 2, 3, 4 or 5 halogen;
G represents 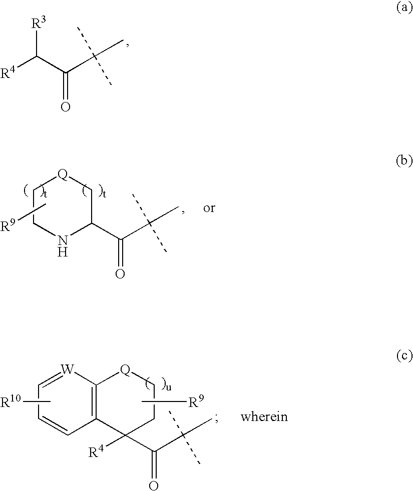
R³ is H, R⁵, C₁₋₆ alkyl, C₂₋₆ alkenyl, C₂₋₆ alkynyl or C₃₋₆ cycloalkyl, wherein each of said C₁₋₆ alkyl, C₂₋₆ alkenyl, C₂₋₆ alkynyl and C₃₋₆ cycloalkyl are independently substituted by 0, 1, 2, 3, 4 or 5 substituents selected from halogen or 0, 1 or 2 substituents selected from OH, oxo, cyano, NH₂, NH(C₁₋₄ alkyl), N(C₁₋₄ alkyl)₂, C₁₋₄ alkyl, C₃₋₆ cycloalkyl, C₄₋₇ cycloalkenyl, cycloheteroalkyl, R⁵ and R⁶;
R⁵ is phenyl, a 5 or 6-membered heteroaromatic ring containing 1, 2 or 3 heteroatoms independently selected from O, S and N, a 4-, 5- or 6-membered cycloheteroalkyl ring containing 1 or 2 heteroatoms independently selected from O, S and N or a phenyl-fused 5- or 6-membered cycloheteroalkyl ring containing 1 or 2 heteroatoms independently selected from O, S and N, wherein said phenyl, said heteroaromatic ring, said cycloheteroalkyl ring and said phenyl-fused cycloheteroalkyl ring are substituted, at any carbon ring atom, by 0, 1, 2, 3, 4 or 5 substituents independently selected from COOH, OH, halogen, CF₃, CHF₂, CH₂F, cyano, C₁₋₆ alkyl, R⁶ and SO₂R⁷;
R⁶ is C₁₋₆ alkoxy, wherein said C₁₋₆ alkoxy is substituted by 0, 1, 2, 3, 4 or 5 halogen;
R⁷ is C₁₋₆ alkyl;
R⁴ is OH or NHR⁸, wherein R⁸ is H or SO₂R⁷ wherein said R⁷ is substituted by 0, 1, 2 or 3 substituents independently selected from OH, halogen, cyano, R⁶ and C₃₋₇ cycloalkyl;
Q is O, CH₂ or S(O)ₙ;
W is C or N;
n is independently 0, 1 or 2;
t is independently 0, 1 or 2;
u is independently 0 or 1;
R⁹ is 0, 1, 2, 3, 4 or 5 substituents selected from halogen, OH, oxo, cyano, C₁₋₄ alkyl, C₃₋₆ cycloalkyl, R⁵ and R⁶, wherein said C₁₋₄ alkyl is substituted by 0 or 1 substituent selected from R⁵, NH₂, NH(C₁₋₄ alkyl) and N(C₁₋₄ alkyl)₂; and
R¹⁰ is 0, 1, 2, 3, 4 or 5 substituents selected from halogen, OH, cyano, C₁₋₄ alkyl, C₃₋₆ cycloalkyl, R⁵ and R⁶, wherein said C₁₋₄ alkyl is substituted by 0 or 1 substituent selected from R⁵, NH₂, NH(C₁₋₄ alkyl) and N(C₁₋₄ alkyl)₂;
or a pharmaceutically acceptable salt or an enantiomer or a pharmaceutically acceptable salt of said enantiomer.

2. A compound according to claim 1, wherein G is

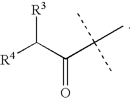

3. A compound according to claim 2, wherein
R¹ is a 5-membered heteroaryl ring containing 2, 3 or 4 heteroatoms, selected from N, O and S, wherein at least 2 heteroatoms are N, and 0 or 1 heteroatoms are O or S;
R² is H or halogen;
R³ is C₁₋₆ alkyl, C₃₋₆ cycloalkyl, a 5 or 6-membered heteroaromatic ring containing 1, 2 or 3 heteroatoms independently selected from O, S and N, a 4-, 5- or 6-membered cycloheteroalkyl ring containing 1 or 2 heteroatoms independently selected from O, S and N, or R¹¹, wherein said C₁₋₆ alkyl, said C₃₋₆ cycloalkyl, said heteroaromatic ring and said cycloheteroalkyl ring are substituted by 0 or 1 substituents selected from NH₂, NH(C₁₋₄ alkyl), N(C₁₋₄ alkyl)₂, C₃ cycloalkyl, R⁶ and R¹¹;
R¹¹ is phenyl, wherein said phenyl is substituted by 0, 1 or 2 substituents selected from halogen and R⁶; and
R⁴ is OH or NH₂.

4. A compound according to claim 3, wherein
R¹ is triazole;
R² is H, Cl or F; and
R³ is C₃₋₆ cycloalkyl, R¹¹ or C₁₋₆ alkyl, wherein said C₁₋₆ alkyl is substituted by 0 or 1 substituents selected from C₃ cycloalkyl, N(C₁₋₄ alkyl)₂, R⁶ and R¹¹.

5. A compound according to claim 3, wherein
R¹ is tetrazole;
R is H, Cl or F; and
R³ is C₃₋₆ cycloalkyl, R¹¹ or C₁₋₆ alkyl, wherein said C₁₋₆ alkyl is substituted by 0 or 1 substituents selected from C₃ cycloalkyl, N(C₁₋₄ alkyl)₂, R⁶ and R¹¹.

6. A compound of formula (X), 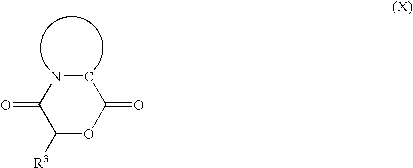
wherein 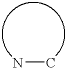
forms an aza-bicyclo[3.1.0]hexane, or 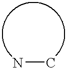
forms an aza-bicyclo[2.1.1]hexane;
R³ is C₁₋₆ alkyl, C₃₋₆ cycloalkyl, a 5 or 6-membered heteroaromatic ring containing 1, 2 or 3 heteroatoms independently selected from O, S and N, a 4-, 5- or 6-membered cycloheteroalkyl ring containing 1 or 2 heteroatoms independently selected from O, S and N, or R¹¹, wherein said C₁₋₆ alkyl, said C₃₋₆ cycloalkyl, said heteroaromatic ring and said cycloheteroalkyl ring are substituted by 0 or 1 substituents selected from NH₂, NH(C₁₋₄ alkyl), N(C₁₋₄ alkyl)₂, C₃ cycloalkyl, R⁶ and R¹¹;
R⁶ is C₁₋₆ alkoxy, wherein said C₁₋₆ alkoxy is substituted by 0, 1, 2, 3, 4 or 5 halogen; and
R¹¹ is phenyl, wherein said phenyl is substituted by 0, 1 or 2 substituents selected from halogen and R⁶.

7. A compound according to claim 3, wherein the stereochemical configuration around the carbon in the aza-bicyclo[3.1.0]hexane or aza-bicyclo[2.1.1]hexane which is covalently bound to the carbonyl is (S) and the stereochemical configuration around the carbon substituted by R³ and R⁴ in G is (R).

8. A compound according to claim 1, wherein G is

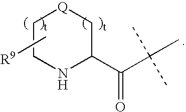

9. A compound according to claim 8, wherein
R¹ is a 5-membered heteroaryl ring containing 2, 3 or 4 heteroatoms, selected from N, O and S, wherein at least 2 heteroatoms are N, and 0 or 1 heteroatoms are O or S;
R² is H or halogen;
R⁹ is 0, 1 or 2 substituents selected from oxo, C₁₋₄ alkyl, R⁵ and R⁶;
R⁵ is phenyl, which is substituted, by 0, 1, 2, 3, 4 or 5 substituents independently selected from COOH, OH, halogen, CF₃, cyano, C₁₋₆ alkyl, R⁶ and SO₂R⁷;
Q is O or CH₂; and
t is independently 0 or 1.

10. A compound according to claim 9, wherein
R¹ is triazole;
R² is H, Cl or F; and
R⁹ is 0, 1 or 2 substituents selected from oxo and C₁₋₄ alkyl.

11. A compound according to claim 9, wherein
R¹ is tetrazole;
R² is H, Cl or F; and
R⁹ is 0, 1 or 2 substituents selected from oxo and C₁₋₄ alkyl.

12. A compound according to claim 9, wherein the stereochemical configuration around the carbon in the aza-bicyclo[3.1.0]hexane or aza-bicyclo[2.1.1]hexane which is covalently bound to the carbonyl is (S).

13. A compound according to claim 1, wherein G is

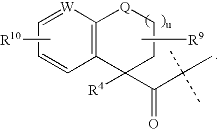

14. A compound according to claim 13, wherein
R¹ is a 5-membered heteroaryl ring containing 2, 3 or 4 heteroatoms, selected from N, O and S, wherein at least 2 heteroatoms are N, and 0 or 1 heteroatoms are O or S;
R² is H or halogen;
R⁴ is OH or NH₂;
R⁹ is 0, 1 or 2 substituents selected from C₁₋₄ alkyl, halogen and R⁶;
R¹⁰ is 0, 1 or 2 substituents selected from C₁₋₄ alkyl, halogen and R⁶;
Q is O or CH₂; and
u is independently 0 or 1.

15. A compound according to claim 14, wherein
R¹ is triazole;
R² is H, Cl or F;
R⁹ is 0, 1 or 2 substituents selected from C₁₋₄ alkyl, F, Cl, OCH₃, OCF₃, OCHF₂ and OCH₂F; and
R¹⁰ is 0, 1 or 2 substituents selected from C₁₋₄ alkyl, F, Cl, OCH₃, OCF₃, OCHF₂ and OCH₂F.

16. A compound according to claim 14, wherein
R¹ is tetrazole;
R² is Cl;
R⁹ is 0, 1 or 2 substituents selected from C₁₋₄ alkyl, F, Cl, OCH₃, OCF₃, OCHF₂ and OCH₂F; and
R¹⁰ is 0, 1 or 2 substituents selected from C₁₋₄ alkyl, F, Cl, OCH₃, OCF₃, OCHF₂ and OCH₂F.

17. A compound of formula (XI), 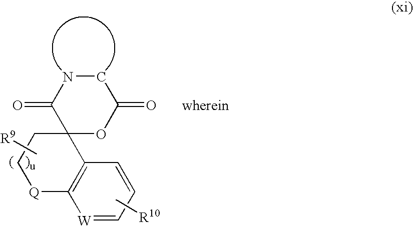 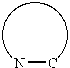
forms an aza-bicyclo[3.1.0]hexane, or 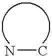
forms an aza-bicyclo[2.1.1]hexane;
R⁹ is 0, 1 or 2 substituents selected from C₁₋₄ alkyl, halogen and R⁶;
R¹⁰ is 0, 1 or 2 substituents selected from C₁₋₄ alkyl, halogen and R⁶;
R⁶ is C₁₋₆ alkoxy, wherein said C₁₋₆ alkoxy is substituted by 0, 1, 2, 3, 4 or 5 halogen;
Q is O or CH₂; and
u is independently 0 or 1.

18. A compound according to claim 14, wherein the stereochemical configuration around the carbon in the aza-bicyclo[3.1.0]hexane or aza-bicyclo[2.1.1]hexane which is covalently bound to the carbonyl is (S).

19. A compound according to claim 1 which is selected from
(1S,2S,5R)-3-((R)-2-Hydroxy-3,3-dimethyl-butyryl)-3-aza-bicyclo[3.1.0]hexane-2-carboxylic acid 5-chloro-2-tetrazol-1-yl-benzylamide,
(1S,2S,5R)-3-((R)-2-Hydroxy-2-phenyl-acetyl)-3-aza-bicyclo[3.1.0]hexane-2-carboxylic acid 5-chloro-2-tetrazol-1-yl-benzylamide,
(1R,2S,5S)-3-((R)-2-Hydroxy-2-phenyl-acetyl)-3-aza-bicyclo[3.1.0]hexane-2-carboxylic acid 5-chloro-2-tetrazol-1-yl-benzylamide,
(1R,3S,5R)-2-((R)-2-Hydroxy-2-phenyl-acetyl)-2-aza-bicyclo[3.1.0]hexane-3-carboxylic acid 5-chloro-2-tetrazol-1-yl-benzylamide,
(1S,3S,5S)-2-((R)-2-Hydroxy-2-phenyl-acetyl)-2-aza-bicyclo[3.1.0]hexane-3-carboxylic acid 5-chloro-2-tetrazol-1-yl-benzylamide,
(1R,2S,5S)-3-((R)-2-Hydroxy-3,3-dimethyl-butyryl)-3-aza-bicyclo[3.1.0]hexane-2-carboxylic acid 5-chloro-2-tetrazol-1-yl-benzylamide,
(1R,3S,5R)-2-((R)-2-Hydroxy-3,3-dimethyl-butyryl)-2-aza-bicyclo[3.1.0]hexane-3-carboxylic acid 5-chloro-2-tetrazol-1-yl-benzylamide,
(1S,3S,5S)-2-((R)-2-Hydroxy-3,3-dimethyl-butyryl)-2-aza-bicyclo[3.1.0]hexane-3-carboxylic acid 5-chloro-2-tetrazol-1-yl-benzylamide,
(1R,2S,5S)-3-(2-Hydroxy-hexanoyl)-3-aza-bicyclo[3.1.0]hexane-2-carboxylic acid 5-chloro-2-tetrazol-1-yl-benzylamide,
(1R,3S,5R)-2-(2-Hydroxy-hexanoyl)-2-aza-bicyclo[3.1.0]hexane-3-carboxylic acid 5-chloro-2-tetrazol-1-yl-benzylamide,
(1S,3S,5S)-2-(2-Hydroxy-hexanoyl)-2-aza-bicyclo[3.1.0]hexane-3-carboxylic acid 5-chloro-2-tetrazol-1-yl-benzylamide,
(1R,3S,5R)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide,
(1S,3S,5S)-2-((R)-2-Hydroxy-4,4-dimethyl-pentanoyl)-2-aza-bicyclo[3.1.0]hexane-3-carboxylic acid 5-chloro-2-tetrazol-1-yl-benzylamide,
(1S,2S,5R)-3-((R)-2-Hydroxy-4,4-dimethyl-pentanoyl)-3-aza-bicyclo[3.1.0]hexane-2-carboxylic acid 5-chloro-2-tetrazol-1-yl-benzylamide,
(1S,2S,5R)-3-((R)-3-Cyclopropyl-2-hydroxy-propionyl)-3-aza-bicyclo[3.1.0]hexane-2-carboxylic acid 5-chloro-2-tetrazol-1-yl-benzylamide,
(1R,3S,5R)-2-((R)-3-Cyclopropyl-2-hydroxy-propionyl)-2-aza-bicyclo[3.1.0]hexane-3-carboxylic acid 5-chloro-2-tetrazol-1-yl-benzylamide,
(1S,3S,5S)-2-((R)-3-Cyclopropyl-2-hydroxy-propionyl)-2-aza-bicyclo[3.1.0]hexane-3-carboxylic acid 5-chloro-2-tetrazol-1-yl-benzylamide,
(1S,2S,5R)-3-((R)-2-Amino-4,4-dimethyl-pentanoyl)-3-aza-bicyclo[3.1.0]hexane-2-carboxylic acid 5-chloro-2-tetrazol-1-yl-benzylamide,
(1R,3S,5R)-2-((R)-2-Amino-4,4-dimethyl-pentanoyl)-2-aza-bicyclo[3.1.0]hexane-3-carboxylic acid 5-chloro-2-tetrazol-1-yl-benzylamide,
(1S,3S,5S)-2-((R)-2-Amino-4,4-dimethyl-pentanoyl)-2-aza-bicyclo[3.1.0]hexane-3-carboxylic acid 5-chloro-2-tetrazol-1-yl-benzylamide,
(1S,2S,5R)-3-((R)-2-Cyclohexyl-2-hydroxy-acetyl)-3-aza-bicyclo[3.1.0]hexane-2-carboxylic acid 5-chloro-2-tetrazol-1-yl-benzylamide,
(1R,3S,5R)-2-((R)-2-Cyclohexyl-2-hydroxy-acetyl)-2-aza-bicyclo[3.1.0]hexane-3-carboxylic acid 5-chloro-2-tetrazol-1-yl-benzylamide,
(1S,2S,5R)-3-((R)-3-tert-Butoxy-2-hydroxy-propionyl)-3-aza-bicyclo[3.1.0]hexane-2-carboxylic acid 5-chloro-2-tetrazol-1-yl-benzylamide,
(1R,3S,5R)-2-((R)-3-tert-Butoxy-2-hydroxy-propionyl)-2-aza-bicyclo[3.1.0]hexane-3-carboxylic acid 5-chloro-2-tetrazol-1-yl-benzylamide,
(1S,2S,5R)-3-((R)-2-Hydroxy-3-phenyl-propionyl)-3-aza-bicyclo[3.1.0]hexane-2-carboxylic acid 5-chloro-2-tetrazol-1-yl-benzylamide,
(1R,2S,5S)-3-((R)-2-Hydroxy-3-phenyl-propionyl)-3-aza-bicyclo[3.1.0]hexane-2-carboxylic acid 5-chloro-2-tetrazol-1-yl-benzylamide,
(1S,5R)-2-((R)-2-Cyclohexyl-2-hydroxy-acetyl)-2-aza-bicyclo[3.1.0]hexane-1-carboxylic acid-5-chloro-2-tetrazol-1-yl-benzylamide,
2-((R)-2-Hydroxy-4,4-dimethyl-pentanoyl)-2-aza-bicyclo[2.1.1]hexane-1-carboxylic acid 5-chloro-2-tetrazol-1-yl-benzylamide,
2-((R)-2-Hydroxy-3-phenyl-propionyl)-2-aza-bicyclo[2.1.1]hexane-1-carboxylic acid 5-chloro-2-tetrazol-1-yl-benzylamide,
2-((R)-2-Hydroxy-2-phenyl-acetyl)-2-aza-bicyclo[2.1.1]hexane-1-carboxylic acid 5-chloro-2-tetrazol-1-yl-benzylamide,
(1S,3S,5S)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1R,2S,5S)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1S,3S,5S)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide,
(1S,2S,5R)-3-[(R)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1S,2S,5R)-3-[2-((R)-3-Chloro-5-difluoromethoxy-phenyl)-2-hydroxy-acetyl]-3-aza-bicyclo[3.1.0]hexane-2-carboxylic acid 5-chloro-2-tetrazol-1-yl-benzylamide,
(1S,3S,5S)-2-[(R)-2-(3-Chloro-5-difluoromethoxy-phenyl)-2-hydroxy-acetyl]-2-aza-bicyclo[3.1.0]hexane-3-carboxylic acid 5-chloro-2-tetrazol-1-yl-benzylamide,
(1S,2S,5R)-3-(2-Hydroxy-hexanoyl)-3-aza-bicyclo[3.1.0]hexane-2-carboxylic acid 5-chloro-2-tetrazol-1-yl-benzylamide,
(1S,3S,5S)-2-((R)-2-Hydroxy-3-pyridin-2-yl-propionyl)-2-aza-bicyclo[3.1.0]hexane-3-carboxylic acid 5-chloro-2-tetrazol-1-yl-benzylamide,
(1R,2S,5S)-3-(2-Hydroxy-3-methoxy-3-methyl-butyryl)-3-aza-bicyclo[3.1.0]hexane-2-carboxylic acid 5-chloro-2-tetrazol-1-yl-benzylamide,
(1S,3S,5S)-2-(2-Hydroxy-3-methoxy-3-methyl-butyryl)-2-aza-bicyclo[3.1.0]hexane-3-carboxylic acid 5-chloro-2-tetrazol-1-yl-benzylamide,
(1S,3S,5S)-2-[(R)-2-(3-Chloro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-((R)-2-Amino-2-phenyl-acetyl)-2-aza-bicyclo[3.1.0]hexane-3-carboxylic acid 5-chloro-2-tetrazol-1-yl-benzylamide,
(1R,2S,5S)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide,
(1R,3S,5R)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide,
(1R,3S,5R)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-[2-(3-Fluoro-phenyl)-2-hydroxy-acetyl]-2-aza-bicyclo[3.1.0]hexane-3-carboxylic acid 5-chloro-2-tetrazol-1-yl-benzylamide,
(1S,3S,5S)-2-[(R)-2-Amino-2-(4-hydroxy-phenyl)-acetyl]-2-aza-bicyclo[3.1.0]hexane-3-carboxylic acid 5-chloro-2-tetrazol-1-yl-benzylamide hydrochloride,
(1S,3S,5S)-2-((R)-2-Amino-3-hydroxy-propionyl)-2-aza-bicyclo[3.1.0]hexane-3-carboxylic acid 5-chloro-2-tetrazol-1-yl-benzylamide,
(1R,3S,5R)-2-((R)-2-Amino-3-hydroxy-propionyl)-2-aza-bicyclo[3.1.0]hexane-3-carboxylic acid 5-chloro-2-tetrazol-1-yl-benzylamide,
(1S,3S,5S)-2-(2-Hydroxy-3-1,2,4-triazol-1-yl-propionyl)-2-aza-bicyclo[3.1.0]hexane-3-carboxylic acid 5-chloro-2-tetrazol-1-yl-benzylamide,
(1S,2S,5R)-3-(2-Hydroxy-3-1,2,4-triazol-1-yl-propionyl)-3-aza-bicyclo[3.1.0]-2-carboxylic acid 5-chloro-2-tetrazol-1-yl-benzylamide,
(1S,3S,5S)-2-((R)-2-Amino-3-tert-butoxy-propionyl)-2-aza-bicyclo[3.1.0]hexane-3-carboxylic acid 5-chloro-2-tetrazol-1-yl-benzylamide,
(1S,2S,5R)-3-((R)-2-Amino-3-tert-butoxy-propionyl)-3-aza-bicyclo[3.1.0]hexane-2-carboxylic acid 5-chloro-2-tetrazol-1-yl-benzylamide,
(1S,2S,5R)-3-[2-(2-Fluoro-phenyl)-2-hydroxy-acetyl]-3-aza-bicyclo[3.1.0]hexane-2-carboxylic acid 5-chloro-2-tetrazol-1-yl-benzylamide,
(1S,3S,5S)-2-[2-(2-Fluoro-phenyl)-2-hydroxy-acetyl]-2-aza-bicyclo[3.1.0]hexane-3-carboxylic acid 5-chloro-2-tetrazol-1-yl-benzylamide,
(1S,2S,5R)-3-[(R)-2-(3-Chloro-phenyl)-2-hydroxy-acetyl]-3-aza-bicyclo[3.1.0]hexane-2-carboxylic acid 5-chloro-2-tetrazol-1-yl-benzylamide,
(1R,2S,5S)-3-[(S)-2-Hydroxy-3-(1-methyl-cyclopropyl)-propionyl]-3-aza-bicyclo[3.1.0]hexane-2-carboxylic acid 5-chloro-2-tetrazol-1-yl-benzylamide,
(1R,3S,5R)-2-[(R)-2-Hydroxy-3-(1-methyl-cyclopropyl)-propionyl]-2-aza-bicyclo[3.1.0]hexane-3-carboxylic acid 5-chloro-2-tetrazol-1-yl-benzylamide,
(1R,3S,5R)-2-(3-Cyclopropyl-2-hydroxy-3-methyl-butyryl)-2-aza-bicyclo[3.1.0]hexane-3-carboxylic acid 5-chloro-2-tetrazol-1-yl-benzylamide,
(1R,2S,5S)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide,
(1S,2S,5R)-3-(3-Cyclopropyl-2-hydroxy-3-methyl-butyryl)-3-aza-bicyclo[3.1.0]hexane-2-carboxylic acid 5-chloro-2-tetrazol-1-yl-benzylamide,
(1S,3S,5S)-2-(4-Hydroxy-1-benzopyran-4-carbonyl)-2-aza-bicyclo[3.1.0]hexane-3-carboxylic acid 5-chloro-2-tetrazol-1-yl-benzylamide,
(rac)-2-(4-Hydroxy-1-benzopyran-4-carbonyl)-2-aza-bicyclo[2.1.1]hexane-1-carboxylic acid 5-chloro-2-tetrazol-1-yl-benzylamide,
2-((R)-2-Hydroxy-4-phenyl-butyryl)-2-aza-bicyclo[2.1.1]hexane-1-carboxylic acid 5-chloro-2-tetrazol-1-yl-benzylamide,
2-((R)-3-tert-Butoxy-2-hydroxy-propionyl)-2-aza-bicyclo[2.1.1]hexane-1-carboxylic acid 5-chloro-2-tetrazol-1-yl-benzylamide,
2-((R)-2-Cyclohexyl-2-hydroxy-acetyl)-2-aza-bicyclo[2.1.1]hexane-1-carboxylic acid 5-chloro-2-tetrazol-1-yl-benzylamide,
2-((R)-2-Amino-3-cyclohexyl-propionyl)-2-aza-bicyclo[2.1.1]hexane-1-carboxylic acid 5-chloro-2-tetrazol-1-yl-benzylamide,
(1S,3S,5S)-2-(2-Hydroxy-3-1,2,4-triazol-1-yl-propionyl)-2-aza-bicyclo[3.1.0]hexane-3-carboxylic acid 5-chloro-2-1,2,4-triazol-1-yl-benzylamide,
(1S,3S,5S)-2-[(R)-2-(3-Chloro-phenyl)-2-hydroxy-acetyl]-2-aza-bicyclo[3.1.0]hexane-3-carboxylic acid 5-fluoro-2-tetrazol-1-yl-benzylamide,
(1S,3S,5S)-2-[(R)-2-Amino-2-(3-chloro-phenyl)-acetyl]-2-aza-bicyclo[3.1.0]hexane-3-carboxylic acid 5-chloro-2-tetrazol-1-yl-benzylamide,
(1S,3S,5S)-2-[2-Amino-2-(1,1-dioxo-hexahydro-1□⁶-thiopyran-4-yl)-acetyl]-2-aza-bicyclo[3.1.0]hexane-3-carboxylic acid 5-chloro-2-tetrazol-1-yl-benzylamide,
(1S,3S,5S)-2-[2-Amino-2-(2-fluoro-phenyl)-acetyl]-2-aza-bicyclo[3.1.0]hexane-3-carboxylic acid 5-chloro-2-tetrazol-1-yl-benzylamide,
(1S,2S,5R)-3-[2-Amino-2-(2-fluoro-phenyl)-acetyl]-3-aza-bicyclo[3.1.0]hexane-2-carboxylic acid 5-chloro-2-tetrazol-1-yl-benzylamide,
(1S,2S,5R)-3-((R)-Morpholine-3-carbonyl)-3-aza-bicyclo[3.1.0]hexane-2-carboxylic acid 5-chloro-2-tetrazol-1-yl-benzylamide,
(1S,3S,5S)-2-(5-Hydroxy-5,6,7,8-tetrahydro-quinoline-5-carbonyl)-2-aza-bicyclo[3.1.0]hexane-3-carboxylic acid 5-chloro-2-tetrazol-1-yl-benzylamide,
2-[(R)-2-Hydroxy-3-(1-methyl-cyclopropyl)-propionyl]-2-aza-bicyclo[2.1.1]hexane-1-carboxylic acid 5-chloro-2-tetrazol-1-yl-benzylamide,
2-((R)-2-Hydroxy-3,3-dimethyl-butyryl)-2-aza-bicyclo[2.1.1]hexane-1-carboxylic acid 5-chloro-2-tetrazol-1-yl-benzylamide,
(1S,5R)-2-((R)-2-Hydroxy-3,3-dimethyl-butyryl)-2-aza-bicyclo[3.1.0]hexane-1-carboxylic acid 5-chloro-2-tetrazol-1-yl-benzylamide,
2-((R)-2-Amino-3,3-dimethyl-butyryl)-2-aza-bicyclo[2.1.1]hexane-1-carboxylic acid 5-chloro-2-tetrazol-1-yl-benzylamide,
(1S,2S,5R)-3-(4-Hydroxy-chroman-4-carbonyl)-3-aza-bicyclo[3.1.0]hexane-2-carboxylic acid 5-chloro-2-tetrazol-1-yl-benzylamide,
(1S,5R)-2-((R)-2-Hydroxy-4,4-dimethyl-pentanoyl)-2-aza-bicyclo[3.1.0]hexane-1-carboxylic acid 5-chloro-2-tetrazol-1-yl-benzylamide,
(1S,5R)-2-((R)-2-Hydroxy-2-phenyl-acetyl)-2-aza-bicyclo[3.1.0]hexane-1-carboxylic acid 5-chloro-2-tetrazol-1-yl-benzylamide,
(1S,3S,5S)-2-((R)-2-Hydroxy-3-pyrazol-1-yl-propionyl)-2-aza-bicyclo[3.1.0]hexane-3-carboxylic acid 5-chloro-2-tetrazol-1-yl-benzylamide,
(1S,2S,5R)-3-((R)-2-Hydroxy-3-pyrazol-1-yl-propionyl)-3-aza-bicyclo[3.1.0]hexane-2-carboxylic acid 5-chloro-2-tetrazol-1-yl-benzylamide,
(1S,2S,5R)-3-(2-Hydroxy-3-pyridin-2-yl-propionyl)-3-aza-bicyclo[3.1.0]hexane-2-carboxylic acid 5-chloro-2-tetrazol-1-yl-benzylamide,
(1S,5R)-2-((R)-3-tert-Butoxy-2-hydroxy-propionyl)-2-aza-bicyclo[3.1.0]hexane-1-carboxylic acid 5-chloro-2-tetrazol-1-yl-benzylamide,
(1S,3S,5S)-2-(4-Hydroxy-chroman-4-carbonyl)-2-aza-bicyclo[3.1.0]hexane-3-carboxylic acid 5-fluoro-2-tetrazol-1-yl-benzylamide,
(1S,2S,5R)-3-((R)-2-Hydroxy-3,3-dimethyl-butyryl)-3-aza-bicyclo[3.1.0]hexane-2-carboxylic acid 5-chloro-2-[1,2,4]triazol-1-yl-benzylamide,
(1S,3S,5S)-2-[(R)-2-Hydroxy-3-(3-methyl-3H-imidazol-4-yl)-propionyl]-2-aza-bicyclo[3.1.0]hexane-3-carboxylic acid 5-chloro-2-tetrazol-1-yl-benzylamide,
(1S,3S,5S)-2-(2-Hydroxy-3-piperidin-4-yl-propionyl)-2-aza-bicyclo[3.1.0]hexane-3-carboxylic acid 5-chloro-2-tetrazol-1-yl-benzylamide and
(1S,3S,5S)-2-((R)-Morpholine-3-carbonyl)-2-aza-bicyclo[3.1.0]hexane-3-carboxylic acid 5-chloro-2-tetrazol-1-yl-benzylamide acetate.

20. A pharmaceutical formulation comprising a compound of formula (I) according to claim 1 in admixture with at least one pharmaceutically acceptable carrier, excipient or diluent.

21-26. (canceled)

27. A method of treatment of a condition where inhibition of thrombin is beneficial, which method comprises administration of a therapeutically effective amount of a compound of claim 1 to a person suffering from, or susceptible to, such a condition.

28. A method of treatment or prevention of a thromboembolic disorder, which method comprises administration of a therapeutically effective amount of a compound of claim 1 to a person suffering from, or susceptible to, a thrombophilia condition.
